(19) United States
(12) Patent Application Publication
(10) Pub. No.: US 2026/0092101 A1
(43) Pub. Date: Apr. 2, 2026

(54) METHOD FOR TREATING SEPSIS AND RELATED DISEASES

(71) Applicant: The Regents of the University of Colorado, a body corporate, Denver, CO (US)

(72) Inventor: Leland Shapiro, Denver, CO (US)

(73) Assignee: The Regents of the University of Colorado, a body corporate, Denver, CO (US)

(21) Appl. No.: 19/347,673

(22) Filed: Oct. 1, 2025

Related U.S. Application Data
(60) Provisional application No. 63/702,238, filed on Oct. 2, 2024.

(51) Int. Cl. C07K 16/12; A61K 38/48; C07K 16/36; C12N 15/115; G01N 33/68

(52) U.S. Cl. CPC C07K 16/12; A61K 38/4886; C07K 16/36; C12N 15/115; C12Y 304/24087; G01N 33/6893; C07K 2317/76; C12N 2310/16; G01N 2333/195

(57) ABSTRACT

The present disclosure provides a method for treating sepsis caused by a microorganism infection. The disclosure also relates to a method for identifying and discovering compounds useful in treating sepsis as well as a method for determining appropriate approaches for treating sepsis. In particular, the methods of the disclosure are based on the discovery by the present inventor that sepsis is caused by exotoxin(s) of microorganism or by constitutive proteins that function as exotoxins as in the case of viruses.

### CROSS-REFERENCE TO RELATED APPLICATIONS

This application claims the priority benefit of U.S. Provisional Application No. 63/702,238, filed Oct. 2, 2024, which is incorporated herein by reference in its entirety.

### FIELD

The present disclosure relates to a method for treating sepsis or a sepsis-like condition in a subject. The disclosure also relates to a method for identifying agents or compounds useful in treating sepsis or sepsis-like clinical conditions as well as a method for determining appropriate treatments for sepsis or sepsis-like clinical conditions.

### BACKGROUND

Sepsis is the disease caused by infections that can progress in severity to cause organ dysfunction which can result in disability or death. Clinical criteria used to define sepsis are reviewed in PMID=28752002 (Gül, F. et al., Turk J. Anaesthesiol. Reanim., “Changing Definitions of Sepsis,” 2017, 45, pp. 129-38). These various definitions have in common identification of an infection or clinical suspicion of an infection along with symptoms or signs thought to be caused by that infection. There is a continuum of clinical symptoms and signs that can range in severity from an influenza-like illness to organ malfunction and death. The current concept that dominates thinking in the pathogenesis or origin of sepsis is that natural infection is followed by an over-exuberant host inflammatory response that can damage or kill the host (infected patient). Since inflammation derived from the host is caused by the production of several molecules that include pro-inflammatory cytokines, this idea that hyper inflammation causes sepsis is often referred to as the “cytokine storm” concept. Accordingly, pharmaceutical companies have invested heavily in developing cytokine antagonists and other inflammation-suppressing measures as a means to reduce or prevent cytokine storm and clinical manifestations of sepsis.

The primary cytokines that are thought to be produced by the host in excessive abundance during infections and that are thought to cause sepsis are tumor necrosis factor alpha (TNF-α or TNFα) and interleukin-1 beta (IL-1β). Many other molecules derived from the infected host have also been implicated as causes of sepsis. Despite an investment of billions of dollars into drug discovery and development and numerous clinical trials designed to cure sepsis using drugs that reduce inflammation, e.g., cytokine antagonists or corticosteroids, none has proven to be effective as a sepsis therapy.

Therefore, the main body of literature suggesting that sepsis is caused by the cytokine storm appears to be incorrect. Evidence of such incorrect hypothesis can be readily recognized in the numerous reports of failed clinical trials in sepsis patients that have used drugs/measures to block cytokine biological activity or that block other purported host-derived substances thought to cause the hyperinflammation of sepsis. These studies have all failed to improve the outcome in patients with sepsis (no reduction in mortality had been observed). More than 100 clinical trials have failed to improve sepsis outcomes in patients, and there have been no positive results that produced any specific sepsis treatment in any clinical trial that has employed appropriate methodology, i.e., randomized controlled clinical trials with adequate blinding and statistical methods. Examples of clinical interventions that have failed to treat sepsis in patients include corticosteroids, cytokine antagonists (inhibitors of TNFα or IL-1β as well as others), nitric oxide inhibitors, platelet-activating factor inhibitors, anticoagulation interventions, statin drugs, antipyretics and non-steroidal anti-inflammatory drugs, physical cooling, endotoxin hemofiltration, phospholipase A2 inhibitors, anti-endotoxin antibodies, anti-Toll-Like Receptor drugs, bradykinin antagonists, albumin, and combinations of these interventions. See TABLE 1. For a thorough review, see Shapiro et al., “Chasing the Ghost: Hyperinflammation Does Not Cause Sepsis,” Front. Pharmacol., 2022 Jun. 23, vol. 13, Article 910516. doi: 10.3389/fphar.2022.910516. PMID: 35814227; PMCID: PMC9260244.

TABLE 1
Clinical trials using anti-inflammation treatments that have failed (28-day
mortality endpoint in nearly all studies)

```
Corticosteroids-
  1. 1984/Sprung CL et al, MEJM, vol 311(18), pages 1137,1143, 1984 [PMID: 6384785]
  2. 1987/Bone RC et al, NEJM, vol 317(11), pages 653-658, 1987 [PMID: 3306374]
  3. 1987/The Veterans Administration Systemic Sepsis Cooperative Study Group, NEJM,
vol 317(11), pages 659-665, 1987 [PMID: 2888017]
  4. 2002/Annane D et al, JAMA, vol vol 288(7), pages 863-871, 2002 [PMID: 12186604]
  5. 2007/Cicarelli DD et al, Sao Paulo Medical Journal, vol 25(4), pages 237-241, 2007:
[PMID: 17992396]
  6. 2008/Sprung CL et al, NEJM, vol 358(2), pages 111-124, 2008 [PMID: 18184957]
  7. 2015/Keh D et al, JAMA, vol 316(17), pages 1775-1785, 2016 [PMID: 27695824]
(Randomized controlled study of hydrocortisone to block progression of severe
SEPSIS to septic shock and 28-day mortality as secondary outcome (No steroid effect
for either outcome))
  8. 2016/Gordon AC et al, JAMA vol 316(5), pages 509-518, 2016 [PMID: 27483065].
Pressors ± hydrocortisone
  9. 2018/Venkatesh B et al, NEJM, vol 378(9), pages 797-808, 2018 [PMID: 29347874]
10. 2018/Annane D et al, NEJM, vol 378(9), pages 809-818, 2018 [PMID: 29490185] (No
30-day mortality effect, but lower mortality at ICU discharge, hospital discharge, 90
days,* and 180 days (90-days = primary outcome).)
11. 2021/Liang H et al, Frontiers in Immunology, vol 12, pages 1-17, 2021 [PMID:
34484209] (Comprehensive systematic review and meta-analysis of N = 50 (12,304
patients) randomized controlled trials using corticosteroids vs sepsis. Outcome =
mortality at 28 days or >60 days.)
Aspirin-
  1. 2016/Kor DJ et al, JAMA, May 15, 2016 (E-pub): PMID: 27179988 (Randomized
controlled study of aspirin prophylaxis to avert ARDS; about 77% of patients with
suspected SEPSIS on enrollment-no mortality difference with aspirin.)
Acetaminophen-
  1. 2015/Young P et al, NEJM, vol 373(23), pages 2215-2224, 2015 [PMID: 26436473]
Ibuprofen-
  1. 1991/Haupt MT et al, Critical Care Medicne, vol 19(11), pages 1339-1347, 1991
[PMID: 1935150]
  2. 1997/Bernard GR et al, NEJM, vol 336(13), pages 912-918, 1997 [PMID: 9070471]
Antipyretic therapy-
  1. 2017/Drewry AM et al, Critical Care Medicine, vol 45(5), pages 806-813, 2013
[PMID: 28221185]. Systematic review and meta-analysis (physical cooling, NSAIDS,
acetaminophen)- 8 randomized trials (1,507 patients) + 6 observational studies (2,058
patients) with SEPSIS and outcome 28 day or hospital mortality (no effect in
randomized or in observational data)
Physical cooling (using water-flow cooling blankets)
  1. 2013/Yang Y-L et al, Chinese Medical Journal, vol 126(10), pages 1809-1813, 2013
[PMID: 23673091] (Increased mortality in experimental group (statistically
significant))
Phospholiopase A2 inhibition-
  1. 2003/Abraham E et al, Critical Care Medicine, vol 31(3), pages 718-728, 2003
[PMID: 12626975]
Anti-endotoxin antibodies-
  1. 1988/Calandra T et al, Journal of Infectious Diseases, vol 158(2), pages 312-319,
1988 [PMID = 3136210]
  2. 1991/Ziegler EJ et al, NEJM, vol 324(7), pages 429-438, 1991 [PMID: 1988827]
  3. 1991/Greenman RL et al, JAMA, vol 266(8), pages 1097-1102, 1991 [PMID:
1865542]
  3. 1994/McCloskey RV et al, Annals of Internal Medicine, vol 121(1), pages 1-5, 1994
[PMID: 8198341]
  4. 1999/Derkx B et al, Clinical Infectious Diseases, vol 28, pages 770-777, 1999 [PMID:
10825037]
Endotoxin hemofiltration-
  1. 2014/Iwagami M et al, Critical Care Medicine, vol 42(5), pages 1187-1193, 2014
[PMID: 24365858]
Bactericidal /Permeability-Increasing Protein (recombinant, rBPI₂₁)-
  1. 2000/Levin M et al, The Lancet, vol 356(9234), pages 961-967, 2000, [PMID:
11041396]
Continuous Veno-venous hemofiltration (low volume)-
  1. 2009/Paten D et al, Critical Care medicine, vol 37(3), pages 803-810, 2009 [PMID:
19237881]
Continuous Veno-venous hemofiltration (high volume)-
  1. Joannes-Boyau O et al, Intensive Care Medicine, vol 39(9), pages 1535-1546, 2013
[PMID: 23740278]
  2. 2020/Yin F et al, Annals of Translational Medicine, vol 8(7), pages 1-10, 2020
[PMID: 32395532]. This reports a meta-analysis of randomized controlled studies in
N = 5 studies (N = 241 subjects) showing no differences between high volume
hemofiltration vs low volume hemofiltration in SEPSIS. Since above study showed no
beneficial effect of low volume hemofiltration it is implied that high volume
hemofiltration is likewise ineffective vs SEPSIS.
Plasma Exchange-
  1. 2014/Rimmer E et al, Critical Care, vol 18(6), pages 1-8, 2014 [PMID: 25527094]
Systematic review and meta-analysis identified 4 randomized controlled trials in
patients with sepsis or septic shock. Overall, no benefit . . . but found benefit if analysis
restricted to adult patients (N = 128) but not in children (N = 66)
Polymyxin B Hemoperfusion-
  1. 2018/Dellinger RP et al, JAMA, vol 320(14), pages 1455-1463, 2018 [PMID:
30304428]
  2. 2015/Payen DM et al, Intensive Care Medicine, vol 41(6), pages 975-984, 2015
[PMID: 25862039]
  3. 2009/Cruz DN et al, JAMA, vol 301(23), pages 2445-2452, 2009 [PMID: 19531784]
CytoSorb ® Extracorporeal Cytokine Hemadsorption-
1. 2017/Schadler D et al, Plos One, vol 12(10), pages 1-18, 2017 [PMID:
29084247].
Randomized controlled OPEN-LABEL study (N = 100, mortality = secondary outcome)
Coupled Plasma Filtration Adsorption-
1. 2014/Livigni S et al, BMJ Open, vol Jan 8, 4(1), pages 1-10, 2014 [PMID: 24401721]
Phospholipid Emulsion-
1. 2009/Dellinger RP et al, Critical Care Medicine, vol 37(11), pages 2029-2038, 2009
[PMID: 19770753]
TLR4 antagonist (synthetic)-
1. 2010/Rice TW et al, Critical Care Medicine, vol 38(8), pages 1-10, 2010 [PMID:
20562702]
  2. 2013/Opal SM et al, JAMA, vol 309(11), pages 1154-1162, 2013 [PMID: 23512062]
Anti-CD14 Monoclonal Antibody IC14-
  1. 2023/Mabrey FL et al, Lancet, vol 93, pages 1-12, 2023 [PMID: 37336058] (Small
RCT with 20 anti-CD14 and 20 controls in hospitalized patients with COVID-19 +
severe pneumonia and oxygen requirement with median SOFA = 2 in both groups. No
effect on time to clinical recovery (=NIH hierarchical composite outcome ≤ 3 out of 8
levels) or mortality at 28 or 60 days)
PAF receptor antagonist-
  1. 1994/Dhainaut JF et al, Critical Care Medicine, vol 22(11), pages 1720-1728,
1994
[PMID: 7956274]
  2. 1998/Dhainaut JF et al, Critical Care Medicine, vol 26(12)), pages 1963-1971, 1998
[PMID: 9875905]
  3. 2000/Suputtamongkol Y et al, Antimicrobial Agents and Chemotherapy, vol 44(3),
pages 693-696, 2000 [PMID: 10681340]
  4. 2000/Vincent JL et al, Critical Care Medicine, vol 28(3), pages 638-642, 2000 [PMID:
10752807]
  5. 2000/Poeze M et al, Shock, vol 14(4), pages 421-428, 2000 [PMID: 11049104]
  6. 2004/Opal S et al, Critical Care Medicine, vol 32(2), pages 332-341, 2004 [PMID:
14758145]
Nitric Oxide (NO) inhibition-
  1. 2004/Lopez A et al, Critical Care Medicine, vol 32(1), pages 21-30, 2004 [PMID:
14707556] (Increased mortality in experimental group (statistically significant))
Bradykinin antagonist (synthetic)-
  1. 1997/Fein AM et al, JAMA, vol 277(6), pages 482-487, 1997 [PMID: 9020273]
Antithrombin-3 (natural)-
  1. 2001/Warren BL et al, JAMA, vol 286(15), pages 1868-1878, 2001 [PMID:
11597289]
  2. 2013/Gando S et al, Critical Care, vol 17(6) R297, pages 1-10, 2013 [PMID:
24342495]
Anticoagulation (recombinant tissue factor pathway inhibitor)- (Hypercoagulability thought to
be associated with excessive inflammation; these anticoagulants also possess anti-
inflammatory effects)
  1. 2001/Abraham E et al, Critical Care Medicine, vol 29(11), pages 2081-2088, 2001
[PMID: 11700399]
  2. 2003/Abraham E et al, JAMA, vol 290(2), pages 238-247, 2003 [PMID: 12851279]
Anticoagulation (recombinant soluble human thrombomodulin)-
  1. 2013/Vincent J-L et al, Critical Care Medicine, vol 41(9), pages 2069-2079, 2013
[PMID: 23979365].
  2. 2014/Yamakawa K et al, Journal of Thrombosis and Hemostasis, vol 13 (4), pages
508-519, 2014 [PMID: 25581687] (Review and meta-analysis of drug approved for
DIC therapy in Japan; included 3 randomized controlled trials (N = 838) showing no 28-
30 day mortality benefit in adult DIC patients with SEPSIS or severe SEPSIS)
3. 2019/Vincent J-L et al, JAMA, vol 321(20), pages 1993-2002, 2019 [PMID:
31104069].
Anticoagulation (heparin)-(Heparin possess anti- inflammatory properties, see, for example,
Anastase-Ravion S et al, Journal of Biomedical Materials Research /Part A, vol 66(2), pages
376-384, 2003 [PMID = 12889008]; Li J-P and Vlodavsky I, Thrombosis and Hemostasis, vol
102, pages 823-828, 2009 [PMID = 19888515]; Corrigan JJ, The Journal of Pediatrics, vol
91(5), pages 695-700, 1966 [PMID = 333074]; and Corrigan JJ and Jordan CM, NEJM, vol
283(15), pages 778-782, 1970 [PMID = 4989565])
  1. 2007/Levi M et al, American Journal of Respiratory and Critical Care Medicine, vol
176, pages 483-490, 2007 [PMID: 17556722] (Randomized assignment to heparin in
patients given activated protein C)
  2. 2009/Jaimes F et al, Critical Care Medicine, vol 37(4), pages 1185-1196), 2009
[PMID: 19242322]
  3. 2015/Zarychanski R et al, Critical Care Medicine, vol 43(3), pages 511-518, 2015
[PMID: 25493972] (Systematic review and meta-analysis (9 randomized controlled
trials with 2,637 patients))
Anticoagulation (recombinant Activated Protein C)-
  1. 2001/Bernard GR et al, NEJM, vol 344(10), pages 699-709, 2001 [PMID: 11236773]
(POSITIVE study- could not be replicated)
  2. 2005/Abraham E et al, NEJM ,vol 353(13), pages 1332-1341, 2005 [PMID:
16192478]
  3. 2007/Nadel et al, LANCET, vol 369, pages 836-843, 2007 [PMID: 17350452]
  4. 2012/Ranieri VM et al, NEJM, vol 366(2), pages 2055-2064, 2012 [PMID: 22616830]
Glycemic Control-
  1. 2001/Van den Berghe G et al, NEJM, vol 345(19), pages 1359-1367, 2001
[PMID:11794168]
  2. 2006/Van den Berghe G et al, NEJM, vol 354(5), pages 449-461, 2006 [PMID:
16452557]
  3. 2008/Brunkhorst FM et al, NEJM, vol 358(2), pages 125-139, 2008
[PMID:18184958]
IL-1 receptor antagonist (recombinant)-
  1. 1994/Fisher CJ et al, Critical Care Medicine, vol 22(1), pages 12-21, 1994 [PMID:
8124953]
  2. 1994/Fisher CJ et al, JAMA, vol 271(23), pages 1836-1843, 1994 [PMID: 8196140]
  3. 1997/Opal SM et al, Critical Care Medicine, vol 25(7), pages 1115-1124, 1997
[PMID: 9233735]
TNF antagonists-
  1. 1993/Fisher CJ et al, Critical Care Medicine, vol 21(3), pages 318-327, 1993 [PMID:
8440099]
  2. 1995/Dhainaut J-F et al, Critical Care Medicne, vol 12(9), pages 1461-1469, 1995
[PMID: 7664546]
  3. 1995/Abraham E et al, JAMA, vol 273(12), pages 934-941, 1995 [PMID: 7884952]
  4. 1996/Fisher CJ et al, NEJM, vol 334(26), pages 1697-1702, 1996 [PMID: 8637514];
this is etanercept (Enbrel®) (Increased mortality in experimental group (statistically
significant))
  5. 1996/Cohen J and Carlet J, Critical Care Medicine, vol 24(9), pages 1431-1440, 1996
[PMID: 8797612]
  6. 1996/Reinhart K et al, Critical Care Medicine, vol 24(5), pages 733-742, 2006 [PMID:
8706447] (Subgroup with IL-6 > 1,000 pg/mL and given highest dose anti-TNF
antibody had lower mortality vs controls.)
  7. 1998/Abraham E, Lancet, vol 351(9107), pages 929-933, 1998 [PMID: 9734938]
  8. 2001/Reinhart K et al, Critical Care Medicine, vol 29(4), pages 765-769, 2001 [PMID:
11373466] (Follow-up study after PMID = 8706447 showed no benefit in sepsis
patients with IL-6 > 1,000 pg/mL)
  9. 2004/Panacek EA et al, Critical Care Medicine, vol 32(11), pages 2173-2182, 2004
[PMID: 15640628] (Reported as POSITIVE study, but restricted to subgroup with IL-
6 > 1,000 and using post-study logistic regression to balance data (both elements
established prospectively))
10. 2006/Rice TW et al, Critical Care Medicine, vol 34(9), pages 2271-2281, 2006
[PMID: 16810105]
11. 2012/Morris PE et al, Critical Care, vol 16(1, R31), pages 1-16, 2012 [PMID:
22340283] (Pilot phase 2a study in N = 70 using ovine polyclonal anti-hTNF Fab
fragment also showed significant neutralization of hTNF in serum by AUC max =
69.7%.)
Janus Kinase (JAK) inhibitors-
  1. 2022/Han MK et al, Lancet, vol 4(5), pages e351-e361, 2022 [PMID: 35368384]
(COVID-19 patients with SOFA ≥ 2 and composite endpoint; 29-day mortality
outcome unaffected)
  2. 2022/Rein L et al, Critical Care Medicine, vol 50(12), pages 1701-1713, 2022 [PMID:
36226977] (COVID-19 ARDS and SOFA ≥ 2 with 28-day mortality primary endpoint)
Tyrosine Kinase Inhibitor- Imatinib-
  1. Atmowihardjo LN et al, Critical Care, vol 27(1):226, pages 1-13, 2023 [PMID:
37291677] (Separately, cytokine inhibition shown in vitro and ex vivo
[PMID = 30336225])
Pentoxifylline (phosphodiesterase inhibitor)-
  1. 2019/Tian J et al, Italian Journal of Pediatrics, vol 45(1), pages 1-7, 2019 [PMID:
31439016]. Systematic review and meta-analysis of 7 randomized controlled trials in
430 patients with NEONATAL SEPSIS, Rationale = pentoxifylline inhibits in vitro
TNF production in human PBMC or alveolar macrophages following LPS stimulation
and suppressed spontaneous TNF production in alveolar macrophages from sarcoidosis
patients (Marques LJ et al, American Journal of Respiratory and Critical Care
Medicine, vol 159(2), pages 508-511, 1999 [PMID = 9927365]).
C5a Antagonist (vilobelimab)-
  1. 2021/Bauer M et al, Critical Care Medicine, vol 3(11), pages 1-10, 2021 [PMID:
34806021]
  2. 2022/Vlaar APJ et al, Lancet, vol 10, pages 1137-1146, 2022 [PMID: 36087611]
(Peculiar study in COVID-19 sepsis where data analysis altered at midpoint of study
directed by FDA to assess results with site stratification (original data analysis plan did
not stratify by site). Endpoint = 28-day mortality was not significant according to
FDA-revised data analysis plan with site stratification. HOWEVER, p < 0.05 for data
NOT stratified by site (=original plan). Really?)
C1-esterase inhibitor (purified human)-
  1. 2002/Caliezi C et al, Critical Care medicine, vol 30(8), pages 1722-1728, 2002
[PMID: 12153783] (Small study in 40 patients did not stop conclusion that drug
improved renal function vs placebo.)
Statins-
  1. 2013/Kruger P et al, American Journal of Respiratory and Critical Care Medicine, Vol
187(7), pages 743-750, 2013 [PMID: 23348980]
  2. 2014/McAuley DF et al, New England Journal of Medline, vol 371(18), pages 1695-
1703, 2014 [PMID: 25268516]
  3. 2014/National Heart, Lung, And Blood Institute ARDS Clinical Trials Network, New
England Journal of Medline, vol 370(23), pages 2191-2200, 2014 [PMID: 24835849]
  4. 2015/Thomas G et al, Minerva Anestesiologica, vol 81(8), pages 921-930, 2015
[PMID: 25690048] (Meta-analysis (4 randomized controlled studies, 1818 adult
patients with severe SEPSIS))
  5. 2015/Deshpande, A et al, American Journal of Medicine, vol 128, pages 410-417,
2015 [PMID: 25526798] (Systematic review and meta-analysis (7 randomized
controlled studies, 1720 adult patients with SEPSIS))
  6. 2016/Quinn M et al, Indian Journal of Critical Care Medicine, vol 20(9), pages 534-
541, 2016 [PMID: 27688630] (Systematic review of 8 randomized controlled studies
(N = 2,275 adults with SEPSIS).)
Lactoferrin (recombinant)-
  1. 2015/Vincent J-L et al, Critical Care Medicine, vol 43(9), pages 1832-1838, 2015
[PMID: 26010687] (Phase 2/3 in adults with recombinant lactoferrin (Talactoferrin
with both antimicrobial and immunomodulatory = anti-inflammatory properties).
Randomized placebo-controlled study with suggestion of elevated mortality with
talactoferrin.)
Albumin-
  1. 2014/Caironi P et al, MEJM, vol 370(15), pages 1412-1421, 2014 [PMID: 24635772]
(Albumin possesses anti-inflammatory and NO-scavenger activities)
Extra-Corporeal Membrane Oxygenation (ECMO)-
  1. 2018/Combes A et al, NEJM vol 378(21), pages 1965-1975, 2018 [PMID:
29791822] . Study in patients with severe ARDS, and 78% had severe sepsis or septic
shock.
Perfusion vs Lactate-Based Resuscitation-
  1. 2019/Hernandez G et al, JAMA, vol 321(7), pages 654-664, 2019: PMID: 30772908
[NOTE: no true control group].
Hydrocortisone + Vitamin C/Ascorbate + Thiamine (HAT)-
  1. 2016/Marik PE et al, Chest, vol 151(6), pages 1229-1238, 2016 [PMID: 27940189].
Substantially reduced mortality compared to non-contemporaneous controls (hospital
mortality = 8.5% vs 40.4% = 79% relative mortality reduction.)
  2. 2020/Fujii T et al, JAMA vol 324(5), pages 423-431, 2020 [PMID: 31950979] NOTE:
Septic shock adults in prospective, randomized, controlled (hydrocortisone alone) trial
that was OPEN LABEL; no effect for outcome = alive and vasopressor-free duration.
SUBGROUPS showed no mortality benefit for combination therapy at days 28. 90, or
in ICU (in fact, mortality slightly increased for intervention for all mortality
assessments). This trial was a response to unusual study by Marik
  3. 2020/Mitchell AB et al, American Journal of Medicine, vol 133(5), pages 635- 638,
2020 [PMID: 31469984]. Another study with similar intervention protocl with non-
contemporaneous controls propensity-score matched showed no mortality benefit
(mortality in ICU or 28 days or 60 days). No change in SOFA score over first 3 daus.
Intervention did show reduced length of stay in OCU or in hospital.
  4. 2020/Chang P et al, Chest, vol 158(1), pages 174-183, 2020 [PMID: 32243943].
Randomized + placebo controlled + single-blinded.
  5. 2020/Mohamed ZU et al, Indian Journal of Critical Care Medicine, vol 24(8), pages
653-661, 2020 [PMID: 33024370]. Randomized, open-label.
  6. 2020/Moskowitz A et al, JAMA, vol 324(7), pages 642-650, 2020 [PMID: 32809003].
Randomized + blinded + placebo.
  7. 2021/Sevransky JE et al, JAMA, vol 325(8), pages 742-750, 2021 [PMID: 33620405].
Randomized + placebo + blinded.
  8. 2021/Somagutta R et al, Acute and Critical Care, vol 36(3), pages 185-200 [PMID:
34185956]. Systematic review + meta-analysis of 15 studies (8 RCT + 7
observational; 67,349 patients)- No benefit in hospital or ICU mortality or hospital
length of stay.
  9. 2023/Mohamed A et al, Shock, vol 59(5), pages 697-701, 2023 [PMID: 36870070].
Small RCT open label with 60-day mortality
Vitamin C-
1. 2022/Lamontagne F et al, NEJM, vol 386(25), pages 2387-2398, 2022 [PMID:
  2. 35704292] (iv Vitamin C in blinded randomized placebo-controlled trial (adults with
sepsis and ≤1 day in ICU and on pressor support = Septic shock) showed INCREASED
primary outcome in Vit C group (death or persistent organ malfunction [ = continued
vasopressor or mechanical ventilation or renal renal replacement therapy] at day 28,
p = 0.001; p = NS in adjusted analysis). Septic shock criteria satisfied in 58%)
2023/LOVIT-COVID Investigators, JAMA, vol 330(18), pages 1745-1759, 2023
[PMID: 37877585] (COVID-19 patients identified with “Sepsis”; SOFA recorded but
unreported)
  3. 2023/Yanase F et al, Critical Care, vol 27(1), pages 1-12, 2023, PMID: 37828547
(Mega- dose sodium ascorbate small study (N = 30) with strong design in adults with
septic shock.)
Thiamine-
  1. 2016/Donnino M et al, Critical Care Medicine, vol 44(2), pages 360-367, 2016,
[PMID: 26771781]
  2. 2023/Moskowitz A et al, American Journal of Respiratory and Critical Cre Medicine,
vol 208(5), pages 570-578, 2023, [PMID: 37364280]
  3. 2023/Sangla F et al, Frontiers in Medicine, vol 13 Sept, pages 1-7, 2023 [PMID:
37780556] (Meta-analysis of 5 randomized controlled trials (RCTs) that includes
Donnino et al.)
Levocarnitine-
  1. 2018/Jones AE et al, JAMA Network Open, vol Dec 7;1(8):e186076, pages 1-12,
2018 [PMID: 30646314]. Septic shock 28d mortality or 24 hr change in SOFA
Trimodulin (polyclonal antibody preparation with IgG/IgM/IgA)-
  1. 2018/Welte T et al, Intensive Care Medicine, vol 44(4), pages 438-448, 2018, [PMID:
29632995] (Randomized controlled Phase 2 trial in patients with severe community-
acquired pneumonia + mechanical ventilation with mortality as secondary outcome.
Likely all subjects met sepsis criteria.)
Intravenous Immunoglobulin (IVIG)-
  1. 2007/Laupland KG et al, Critical Care Medicine, vol 35(123), pages 2686-2692, 2007,
[PMID: 18074465] (Rationale = cytokine + pathogen elimination; anti-inflammation
highlighted. Systematic review + meta-analysis of N = 14 randomized controlled trials
showed in adults with SEPSIS showed overall mortality BENEFIT. BUT, mortality
benefit disappeared considering only studies with high methodological quality (N = 4;
allocation concealment + blinding + ITT) or with blinding (N = 6) or with albumin
placebo (N = 7). Also, publication bias strongly suggested)
  2. 2013/Toth I et al, Journal of Anesthesiology, vol 27, pages 618-622, 2013,
[PMID = 23314695] (IgM-enriched Ig used in randomized controlled (partially blinded)
trial in adults (N = 33) with septic shock.)
  3. 2016/Busani S et al, Minerva Anestesiologica, vol 82(5), pages 559-572, 2016, 2016,
[PMID: 26474267] (Meta-analysis in N = 18 randomized controlled trials (2,333
patients) of adults given polyclonal Ig in SEPSIS. Substantial design variability.
Again overall analysis showed mortality reduction = 50% (p = 0.022). HOWEVER,
mortality benefit not observed in pooled studies with mortality measured at ≤28 days
(N- 12 studies), and benefit vanishes in studies with better (adequate) design. No
effect if adequate blinding (N = 5 studies and 852 patients) or in studies published after
year 2000 (N = 8 studies or 1,732 patients))
Neutrophil Elastase Inhibitor-
  1. 2004/Zeiher BG et al, Critical Care Medicine, vol 32(8), pages 1695-1702, 2004,
[PMID: 15286546] (ONO small molecule inhibitor (sivelestat, randomized placebo-
controlled double-blind study) in patients with acute lung injury all on mechanical
ventilation (N = 487), of whom 58.5% were caused by infection. Primary outcome of
28-day all-cause mortality p = NS. Subgroup with pulmonary infection showed no
mortality benefit. Increased mortality in sivelestat group at 180 days by Cochran-
Mantel-Haenszel analysis (p = 0.006); study stopped early.)
Vitamin A-
  1. 2019/Cherukuri L et al, Clinical Nutrition ESPEN, vol 29, pages 49-51, 2019, [PMID:
30661700] (Prospective, randomized, double-blind placebo-controlled trial in patients
with severe sepsis. Small N = 63)
Selenium-
  1. 2019/Li S et al, MEDICINE, vol 98(9), pages 1-7, 2019 [PMID: 30817625] (Meta-
analysis of 13 randomized controlled trials (N = 1,922 patients) of iv Se for 7-21 days
vs SIRS/SEPSIS/Severe SEPSIS/Septic shock in adults. Se = antioxidant)
Beta-Blockers- Landiolol (b-blocker; short-acting)
  1. 2023/Whitehouse et al, JAMA, vol 330(17), pages 1641-1652, 2023, [PMID:
37877587] (Stopped early for futility and possible elevated mortality in septic shock
pateints. Primary outcome = SOFA at 14 days and no difference. Also, no difference
in 30- or 90-day mortalities. Rationale included suppression of
cytokines/inflammation.)
Intravenous fluid RESTRICTION-
  1. 2022/Meyhoff TS et al, NEJM, vol 386(26), pages 2459-2470, 2022, [PMID:
35709019]
Macrolide Antibiotcs- (Used in sepsis for anti-inflammation properties)
  1. 2014/Giamarellos-Bourboulis EJ et al, Journal of Antimicrobial Chemotherapy, vol
69(4), pages 1111-1118, 2014, [PMID: 24292991].
  2. 2022/Karakike E et al, Critical Care, vol 26(1), pages 1-11, 2022, [PMID: 35717241]
  3. 2024/Trifi A et al, Journal of Critical Care, vol 14 Feb, pages 1-7, 2024, [PMID:
38359518]
Triggering Receptor Expressed on Myeloid Cells (TREM)-1; Nangibotide (Nangibotide = 12
amino acid peptide that binds TREM-1 ligand)-
  1. 2020/Francois B et al, Intensive Care Medicine, vol 46(7), pages 1425-1437, 2020,
[PMID: 32468087]
  2. 2023/Francois B et al, Lancet Respiratory Medicine, vol 11(10), pages 894-904, 2023,
[PMID: 37269870]
  3. 2023/Francois B et al, eClincalMedicine (Lancet), vol 60(June 2023), pages 894-904,
2023, [PMID: 37350989] (Severe COVID-19 patients; stopped early (low recruitment)
with mortality as secondary outcome)
Opal et al in 2014: “Hundreds of millions of dollars have been expended enrolling over 30,000 patients in clinical trials to test and develop new immunomodulating agents, anti-inflammatory agents, and antiendotoxin agents. Yet, not a single agent has convincingly proven to be consistently efficacious in clinical trials. There are no new drugs on the market to show for all this effort.” Opal SM et al, Critical Care Medicine, vol 42(7), pages 1714-1721, 2014: PMID = 24717456
Marik et all in 2016: “Over the last 3 decades, more than 100 phase 2 and phase 3 clinical trials have been performed testing various novel pharmacologic agents and therapeutic interventions in an attempt to improve the outcome of patients with severe sepsis and septic shock; all of these efforts ultimately failed to produce a novel pharmacologic agent that improved the outcome of sepsis.” Marik PE et al, Chest, Dec 6 E-pub, 2016: PMID = 27940189
```

Accordingly, in order to treat sepsis and sepsis-like clinical conditions, a better understanding of the underlying cause of sepsis is required. Such an understanding can lead to a proper treatment and/or prophylaxis of sepsis, and development of therapeutic agents, compounds, or active pharmaceutical ingredients (“APIs”) that are effective in (i) treating sepsis, (2) preventing occurrence of sepsis, (3) or both.

### SUMMARY

Some aspects of the present disclosure are based on the discovery by the present inventor of heretofore unrecognized or unknown underlying causes of sepsis and sepsis-like conditions due to microorganism infection. This discovery by the present inventor of the underlying complete causal pathway of sepsis can be used to treat a subject with active sepsis or as a prophylactic measure to block sepsis occurrence in high-risk populations. In general, methods of the disclosure can be used to treat, reduce, or prevent sepsis or any other wide variety of clinical conditions that may result from a microorganism infection.

Thus, one aspect of the discovery provides a method for treating a subject for a clinical condition associated with a microorganism infection. Unlike conventional methods, methods of the present disclosure include administering to a subject in need of such a treatment a therapeutically effective amount of a therapeutic agent capable of reducing the effect of an exotoxin produced by said microorganism or neutralizing the effect of a constitutive molecule that functions as an exotoxin (e.g., as occurs in viruses), thereby treating said subject. Methods of the disclosure can also include administering to a subject in need of such a treatment a therapeutic agent capable of interrupting the pathway involved in sepsis clinical pathogenesis that is caused by exposure to exotoxins or to molecules that function as exotoxins (e.g., viruses themselves). This pathway involves generation of disseminated microthrombi in the body that is a direct consequence of the presence of exotoxins or of constitutive molecules with exotoxin biological activity (e.g., viruses). In some embodiments, said microorganism is a bacterium. Yet in other embodiments, said microorganism is a mycobacterium. Still in other embodiments, said microorganism is a virus. In further embodiments, said microorganism is a fungus. Yet still in other embodiments, said microorganism is a protozoan.

Since some aspects of the present disclosure provide molecular interactions that cause organ malfunction or death resulting from sepsis, many targets are identified that can be engaged or interrupted to treat proximal and downstream detrimental effects of sepsis or other clinical conditions associated with a microorganism infection. In some aspects of the disclosure, exotoxins or of constitutive molecules with exotoxin biological activity are therapeutic targets. Exemplary exotoxins include, but are not limited to, classical or known exotoxins secreted by bacteria as nearly all bacteria produce exotoxins. In addition, as disclosed herein, viruses also employ exotoxins to subvert immunity as a means to promote viral survival. Viruses have evolved to incorporate molecules into their surfaces or internally that function similar to exotoxins secreted by bacteria. This is indicated by noting that “the virus itself is the exotoxin”. Therefore, unless context requires otherwise it is intended that the term “exotoxin” includes both bacterial secreted exotoxins and exotoxin-functioning molecules (i.e., “constitutive molecules with exotoxin biological activity” or “constitutive molecule that functions as an exotoxin”) that are part and parcel of viruses. The biological activities of exotoxins relevant for this disclosure include, for example, the following 3 actions:
1. Exotoxins suppress host (humans or other mammals) innate immune function, and this is the prime activity of these molecules that permits successful infection of the human or other mammalian host. Immune suppressing actions of exotoxins include blockade of cell protein synthesis that prevents the host from generating molecules that coordinate and amplify host response to the pathogen that is designed to kill the pathogen. It is well-described that exotoxins suppress production of host cytokines which are key molecules that amplify immunity. Exotoxins also directly disable immunocytes or cells designed to kill invading pathogens. In this regard, exotoxins depress activities of neutrophils, monocytes, and/or lymphocytes and this promotes the chance of the pathogen to evade killing by immune system of host cells.
2. Exotoxins may also induce disruption of epithelial and/or endothelial barriers. Without being bound by any theory, it is believed that this disruption causes leakiness that permits fluids (especially blood) to seep between the cells of the vasculature and permits substances to leak into the bloodstream. This leakiness fosters transmigration of exotoxins that reside within the subject's gut to gain access to the blood circulation and enhance the net exotoxin detrimental (to the host) biological activities. It has been shown that gut contents contain pre-formed exotoxins that are tolerated due to impermeability of the gut cell barrier. For example, Clostrioides difficile exotoxin is measured in stool of persons with no disease in PMID=29788296 (Pollock et al., Clin Infect Dis., “Comparison of Clostridioides difficile Stool Toxin Concentrations in Adults With Symptomatic Infection and Asymptomatic Carriage Using an Ultrasensitive Quantitative Immunoassay,” 2019 Jan. 1, 68(1), pp. 78-86).
3. Exotoxins induce death of host cells by inducing apoptosis (programmed cell death) after contact with host cells. This is yet another mechanism used by exotoxins to degrade host immunity by eliminating host immunocytes that would otherwise attempt to kill the pathogen.

In other embodiments, said clinical condition associated with a microorganism comprises sepsis or severe sepsis or septic shock, trauma, blood loss, typhlitis, Clostridioides difficile disease, Shiga-like toxin mediated diarrhea, hemolytic-uremic syndrome (HUS), immune reconstitution inflammatory syndrome, disseminated intravascular coagulopathy (DIC), purpura fulminans, necrotizing fasciitis, hemorrhagic fevers, COVID-19, heatstroke, and the like.

Yet in other embodiments, said therapeutic agent comprises an antibody directed against said exotoxin, antitoxin neutralizing resin or other natural or synthetic agent, anti-serum containing an antibody directed against said exotoxin, recombinant ADAMTS-13 (A Disintegrin and Metalloprotease with a Thrombospondin-1 Motif member 13), a plasma composition comprising ADAMTS-13 or natural or synthetic substance with ADAMTS-13 protease activity, an inhibitor that reduces production of or inhibits binding of von Willebrand Factor (vWF) strands to the surface of endothelial cell P-selectin, an inhibitor that reduces or inhibits binding of platelets to vWF strands (e.g., CAPLACIZUMAB, trade name Cablivi®), an aptamer that selectively binds to and inactivates or clears said exotoxin, or a combination thereof. Still in other embodiments, said therapeutic agent comprises an antagonist or an inhibitor of a cell-surface or intracellular receptor that binds with said exotoxin or with a constitutive molecule that functions as an exotoxin, e.g., a protein component of viruses. This approach blocks the ability of the exotoxin or similar viral protein from transmitting the cascade of events that can eventuate in organ malfunction or death. Yet in other embodiments, said therapeutic agent comprises an antibody directed against said exotoxin or binds a pathogen-associated constitutive molecule(s) that functions as an exotoxin. In other embodiments, said therapeutic agent increases anti-microbial immune function by increasing the level of or the activity of a Toll-Like Receptor (TLR), inflammasome, retinoic-acid inducible gene 1 (RIG 1)-like receptor, C-type-lectin-like receptor, or a combination thereof (this is in fact the opposite approach compared to current standard practice and highlights novelty of the present disclosure).

Another aspect of the disclosure provides a method for treating a disease or a clinical condition caused by a microorganism infection in a patient. The method includes:
identifying the microorganism causing the disease or the clinical condition in the patient; and
administering to the patient a therapeutically effective amount of a therapeutic agent that is capable of reducing the effect of one or more exotoxin(s) produced by said microorganism or reducing activity of a constitutive molecule(s) that functions as an exotoxin (viruses),
   thereby treating the disease or the clinical conditions caused by the microorganism infection in the patient. It is known that many pathogens possess several exotoxins or exotoxin-functioning molecules (viruses). Therapeutic benefit can be achieved by neutralizing only one or a few of these toxins.

In some embodiments, said therapeutic agent comprises an antibody directed against said exotoxin, antitoxin resin or other natural or synthetic inhibitor, anti-serum containing an antibody directed against said exotoxin, recombinant ADAMTS-13 or natural or synthetic molecule with ADAMTS-13 biological activity, a plasma composition comprising ADAMTS-13, an inhibitor that reduces synthesis or secretion of vWF strands or inhibits binding of von Willebrand Factor (vWF) to the blood-contacting surface of endothelial cell P-selectin, an inhibitor that reduces binding of platelets to vWF strands, an aptamer that selectively binds to and inactivates said exotoxin, or a combination thereof. A further embodiment includes a measure that reduces production of cell-surface vWF strands.

Still in other embodiments, said clinical condition associated with a microorganism comprises sepsis or severe sepsis or septic shock, trauma, blood loss, typhlitis, Clostridioides difficile disease, Shiga-like toxin mediated diarrhea, hemolytic-uremic syndrome, immune reconstitution inflammatory syndrome, disseminated intravascular coagulopathy (DIC), purpura fulminans, necrotizing fasciitis, hemorrhagic fevers, COVID-19, heatstroke, and the like.

Yet other aspects of the disclosure provide a method for producing a therapeutic agent for treating a microorganism infection in a subject. The method comprises:
identifying one or more exotoxin(s) produced by the microorganism or identifying one or more constitutive microorganism component molecule(s) that possesses exotoxin function (viruses); and
producing an antibody to said exotoxin or producing an aptamer that inhibits activity of said exotoxin(s) or inhibits constitutive microorganism component molecule(s) that possesses exotoxin function. It is noted that most bacteria pathogenic to humans produce more than 1 exotoxin or contain more than one constitutive exotoxin-like molecule (e.g., in viruses). It is the subject of empirical discovery which of these toxins are optimal targets for neutralization (in some cases neutralization of only 1 exotoxin is needed for near complete therapeutic benefit; see for example the use of the Tdap (Tetanus, Diphtheria, acellular Pertussis) vaccine as discussed infra.

In some embodiments, said antibody preparation is a monoclonal antibody. Yet in other embodiments, said antibody preparation is a polyclonal antibody.

Still in other embodiments, said microorganism comprises a bacterium, a mycobacterium, a protozoan, a virus, a fungus, or a combination thereof.

Further aspects of the disclosure provide a method for reducing or preventing a post-operative microorganism infection in a subject. The method comprises administering to a subject who is scheduled for an operation a therapeutically effective amount of a therapeutic agent capable of reducing biological activity of an exotoxin or of a constitutive molecule(s) with exotoxin activity produced by a microorganism that is present within a facility of scheduled operation or recovery. The therapeutic agent is administered to the subject (e.g., enterally, parenterally, or topically) prior to the operation. In a further prophylactic embodiment, any measure designed to inhibit a sepsis-causing molecule (exotoxin or exotoxin-functioning constitutive molecule or vWF strands) or designed to augment ADAMTS-13 bioactivity can be administered empirically to a person suffering significant trauma in the field or in the hospital.

In some embodiments, said microorganism comprises a bacterium, a mycobacterium, a protozoan, a virus, a fungus, or a combination thereof.

Still in other embodiments, said microorganism comprises:
(a) any Gram-positive coccus (aerobic or anaerobic), including Pneumococcus (Streptococcus pneumoniae), Streptococcus groups A, B, C, or G, Streptococcus anginosus group (formerly Streptococcus milleri group=Streptococcus constellatus, Streptococcus intermedius, Streptococcus anginosus), Enterococcus faecalis and Enterococcus faecium, Staphylococci (coagulase negative and coagulase positive bacteria) including methicillin-sensitive Staphylococcus aureus (MSSA), methicillin-resistant Staphylococcus aureus (MRSA), vancomycin-resistant Staphylococcus aureus (VRSA), Staphylococcus epidermidis, Staphylococcus hominis, Leuconostoc;
(b) any Gram-positive rod or bacillus (aerobic or anaerobic), including Clostridia species (C. tetani or C. boltulinum, C sordellii, or C. difficile), Listeria monocytogenes, Erysepelothrix, Bacillus species (Bacillus anthracis, Bacillus cereus);
(c) any Gram-negative coccus (aerobic or anaerobic), including Neisseriae, including Neisseria gonorrhea and Neisseria meningitis, Veillonella, Bartonella species including Bartonella henselae and Bartonella quintana, Moraxella catarrhalis;
(d) any Gram-negative rod or bacillus (aerobic or anaerobic) enteric or non-enteric pathogens including Pseudomonas species, E. coli, Salmonella, Shigella, Campylobacter, Helicobacter pylori and Helicobacter cneidi, Proteus species including Proteus vulgaris and P. rettgeri, Eikenella corrodens, Aggegatibacter actinomycetemcomitans, Coxiella burnetti, Cardiobacterium hominis, Kingella kingae, Bacteroides fragilis, Serratia marcessans, Enterobacter cloacae, Citrobacter freundii, Morganella morganii, Providentia species, Haemophilus influenzae, Capnocytophaga canimorsus, Fusobacterium necrophorum and Fusobacterium nucleatum, Stenotrophomonas maltophilia, Streptobacillus moniliformis, Yersinia species;
(e) any mycobacterium including Mycobacterium tuberculosis (TB) and non-tuberculous mycobacteria like Mycobacteria abscessus/chelonae/fortuitum/chimerae/bovis, Mycobacterium ulcerans, Mycobacterium marinum, Mycobacterium gordonae, Mycobacterium leprae.
(f) mycoplasmas including Mycoplasma pneumoniae, Mycoplasma hominis
(g) Actinomyces species including Actinomyces israelii/constellatus/naeslundii and Nocardia species:
(h) any protozoan pathogen including, Giardia lamblia, Entameba histolytica, Leishmania species (including L. donovani) Malaria (including species Malariae/Ovale/Falciparum/Vivax):
(i) any fungus including Candida (several species including albicans/tropicalis/glabrata/auris).
(j) any mold including Aspergillus (several species like fumigatus, flavus, niger, terreus), Mucorales, Rhizupus:
(k) dimorphic yeasts/molds including Histoplasma capsulatum, Coccidioides species (C. immitis or C. posadasii), Blastomyces dermatitidis, Paracoccidioides species.
(l) any pathogenic virus including: Influenza viruses A, B, C, or D, Cytomegalovirus, Epstein-Bar Virus, Herpes viruses including Herpes simplex 1 and 2, Varicella-Zoster virus, Human Herpes virus 6, Severe Acute Respiratory Syndrome Coronavirus 2 (SARS-COV-2, agent that causes COVID-19), Ebola, Dengue, West Nile Virus, Respiratory syncytial virus, Parvovirus, Measles virus, BK virus, Crimean-Congo virus, Hantaviruses (including sin nombre), Human adenoviruses, JC virus;
(m) or a combination thereof.

It should be understood that the above list in no way restricts the scope of the present disclosure. The scope of the present disclosure is applicable to any microorganism that can cause infection and manifest subsequent clinical symptoms or signs. The discovery that nearly all microorganisms produce organ malfunction or death in infected humans using the same mechanisms is a pivotal discovery of the inventor. In fact, the only human pathogens that are known to not contain a secreted exotoxin or contain a constitutive component molecule(s) with exotoxin bioactivity include spirochetes (bacteria that cause syphilis, Lyme disease, and a cause of relapsing fever).

In some embodiments, said therapeutic agent is administered 1-7 days before the operation or following severe trauma.

Still in other embodiments, said therapeutic agent comprises an antibody to said exotoxin or a constitutive molecule with exotoxin activity (as in the case for viruses).

Further aspects of the disclosure provide a method for antisepsis pre-exposure or post-exposure prophylaxis—this includes:
a. Vaccination given before sepsis (pre-exposure prophylaxis) or given during the early stages of sepsis (post-exposure prophylaxis) prepared by any available state of the art methods that neutralizes exotoxin activities through induction of toxin-neutralizing antibodies or antibodies that block biological activities of constitutive molecules that function as exotoxins (e.g., viral proteins). The concept of post-exposure prophylaxis or PEP is accepted art. It is used for the treatment of patients after rabies exposure where vaccination is given after exposure as an attempt to generate an immune response before disease can initiate. Also, PEP is used routinely after a high-risk event (sexual encounter or transfusion) predisposes to HIV exposure where anti-HIV drugs are given immediately after exposure. Use of HIV PEP has been shown to substantially reduce the risk of HIV infection (see CDC guidelines and description: https://hivinfo.nih.gov/understanding-hiv/fact-sheets/post-exposure-prophylaxis-pep#:˜:text=Post %2Dexposure%20prophylaxis%20(PEP)%20means%20taking %20HIV%20medicines%20within,to%20HIV%20to%20prevent%20HIV.&text=PEP%20must%20be%20started%20within,Every%20hour%20counts).
b. A separate method is administration of parenteral antibody preparations that have exotoxin-neutralizing activities in clinical situations where a patient is at high risk for sepsis. Appropriate exotoxins can be targeted based on the known epidemiology of infections that occur in specific situations. For example, in hospitalized patients at high risk for infection or planned to undergo surgery, parenteral preparations designed to neutralize the most relevant toxins that produce disease originating from Staphylococcus aureus and from Pseudomonas aeruginosa can be used. This should not be limited to antibodies, as any other natural or synthetic substance(s) can be used that functions to block exotoxin activities (resin or designed substances like aptamers, etc.). Note that designed substances have potential for oral, topical, or parenteral use.
c. In some embodiments, this method can be used in conjunction with antibiotics, as is the current standard of care before any invasive surgical procedure. Also note that the use of exotoxin-thwarting substances as proposed in this disclosure and use of prophylactic antibiotics may be synergistic and have a substantial effect in blocking subsequent infection and disease.

It should be appreciated that the list provided herein in no way restricts applicability of this disclosure to microorganisms listed herein. In fact, methods of the disclosure can also be used to other known human pathogens that do not contain a secreted exotoxin or contain a constitutive component molecule(s) with exotoxin bioactivity, such as spirochetes, e.g., bacteria that cause syphilis, Lyme disease, and a cause of relapsing fever.

In some embodiments, prophylaxis of sepsis or a clinical condition associated with microorganism infection include administering a vaccine that promotes immune response to microorganism exotoxin(s). In some embodiments, said vaccine is administered 1-7 days before the operation to prevent or reduce the likelihood of post-operative sepsis or a clinical condition associated with microorganism infection.

Still in other embodiments, prophylaxis or treatment of sepsis or a clinical condition associated with microorganism infection comprises administering an antibody to said exotoxin(s) or one or more constitutive molecule(s) with exotoxin activity.

Other aspects of the disclosure include methods for a therapy that is directed against the biological pathway that causes organ malfunction or death, such as a treatment directed against formation of endothelial cell-surface long von Willebrand Factor (vWF) strands or formation of vWF strands conjoined to circulating platelets. Exemplary therapeutic agents for such methods include, but are not limited to, (i) Recombinant ADAMTS-13 (A Disintegrin and Metalloprotease with a Thrombospondin-1 Motif member 13), a plasma composition comprising ADAMTS-13 or natural or synthetic substance with ADAMTS-13 protease activity, or any blood-derived product that is enriched for biologically active ADAMTS-13; (ii) Administration of a parenteral method that contains a protease that reduces or inhibits attachment of vWF to the blood-interfacing surface of endothelial cell P-selectin, an inhibitor that blocks vWF engagement of P-selectin or suppresses expression of extracellular vWF strands, or interrupts vWF strand attachment to endothelial cells; (iii) an inhibitor that reduces or blocks binding of platelets to vWF strands (for example, CAPLACIZUMAB or CABLIV®), an aptamer that selectively binds to, inactivates, or clears said exotoxin; or (iv) a combination thereof.

Methods disclosed herein can also be used as components of combination (“cocktail”) therapy. Since methods disclosed herein for treating sepsis differs from conventional treatments, use of combined therapies that employ therapies described in this disclosure in addition to traditional antisepsis therapies can also be used. Such a combination therapy includes using traditional therapies with exotoxin neutralization or interruption directed to the biological pathway causing organ damage or death. Therefore, any of the above uses of anti-exotoxin therapies can be used with antibiotics, antiviral measures, and supportive care to maximize patient outcomes. Combination therapies include, but are not limited to, (i) Standard direct antimicrobial drugs like antibiotics, antivirals, antifungal agents, or anti-protozoal drugs; (ii) Standard supportive treatments that include mechanical ventilation, anticoagulation drugs, fluid and electrolyte replenishment, sedatives, as well as other non-specific measures; (iii) Agents/drugs that enhance activity of the innate immune system or pro-inflammation agents such as cytokines or growth factors; (iv) Antibiotics or other agents that suppress protein synthesis of bacteria that reduce the synthesis of exotoxins (often referred to as non-bactericidal antibiotics); (v) Post-sepsis immunization against exotoxins or against viral exotoxin-functioning constitutive components. This constitutes post-exposure prophylaxis or PEP, as used to treat patients after rabies exposure or after HIV exposure; (vi) Exotoxin-neutralizing substances as disclosed herein; (vii) a method as disclosed herein that blocks formation of endothelial cell-surface long vWF strands; and (viii) a method as described herein that blocks formation of endothelial cell-surface long vWF strands conjoined to platelets by interrupting platelet-vWF interactions.

### BRIEF DESCRIPTION OF THE DRAWINGS

The patent or application file contains at least one drawing executed in color. Copies of this patent or patent application publication with color drawing(s) will be provided by the Office upon request and payment of the necessary fec.

FIG. 1 is a schematic illustration of bacterial sepsis pathogenesis.

FIG. 2 is a schematic illustration of viral sepsis pathogenesis.

FIG. 3 is an illustration showing vWF and ADAMTS-13 in Homeostasis.

FIG. 4 is an illustration showing vWF and ADAMTS-13 in Hemostasis.

FIG. 5 is an illustration showing vWF and ADAMTS-13 roles in Immunothrombosis.

FIG. 6 is an illustration showing vWF and ADAMTS-13 roles in sepsis-induced organ malfunction or death.

### DETAILED DESCRIPTION

The present disclosure is described herein with sufficient details to provide an understanding of one or more particular embodiments of broader subject matters. The descriptions expound upon and exemplify features of those embodiments without limiting the scope of disclosure to the explicitly described embodiments and features. Considerations in view of these descriptions will give rise to additional and similar embodiments and features without departing from the scope of the presently disclosed subject matter.

The terms “comprise(s),” “include(s),” “having,” “has,” “can,” “contain(s),” and variants thereof, as used herein, are intended to be open-ended transitional phrases, terms, or words that do not preclude the possibility of additional steps or components or ingredients. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not. Accordingly, the transitional phrases “consisting of” and “consisting essentially of” may be interpreted to be subsets of the open-ended transitional phrases, such as “comprising” and “including,” such that any use of an open-ended phrase to introduce a recitation of a series of elements, limitations, components, ingredients, materials, or steps should be interpreted to also disclose recitation of the series of elements, limitations, components, ingredients, materials, or steps using the closed terms “consisting of” and “consisting essentially of.” For example, the recitation of a composition “comprising” components A, B and C should be interpreted as also disclosing a composition “consisting of” components A, B, and C as well as a composition “consisting essentially of” components A, B, and C.

The terms “treat,” “treating,” “treatment,” and the like refer to eliminating, reducing, relieving, reversing, and/or ameliorating a disease or condition and/or symptoms associated therewith. Although not precluded, treating a disease or condition does not require that the disease, condition, or symptoms associated therewith be completely eliminated, including the treatment of acute or chronic signs, symptoms and/or malfunctions. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition, “treatment” therefore also includes relapse prophylaxis or phase prophylaxis. The term “treat” and synonyms contemplate administering a therapeutically effective amount of a compound of the disclosure to an individual, e.g., a mammalian patient including, but not limited to, humans and veterinary animals, in need of such treatment. A treatment can be orientated symptomatically, for example, to suppress symptoms. It can be affected over a short period, be oriented over a medium term, or can be a long-term treatment, for example within the context of a maintenance therapy.

For the purposes of this disclosure, the terms “administration” or “administering” refer to a method of giving a dosage of a compound or pharmaceutical composition to a subject. A composition described herein may be administered to a subject by any one of a variety of manners or a combination of varieties of manners. For example, a composition may be administered orally, nasally, intraperitoneally, or parenterally, by intravenous, intramuscular, topical, or subcutaneous routes, or by injection into tissue.

Some aspects of the disclosure are based at least in part on the discovery by the present inventor that there is no evidence showing sepsis is caused by or results in excessive inflammation, i.e., cytokine storm. In fact, the present inventor has discovered that sepsis is a disease characterized by profound suppression of host inflammation or cytokine levels (compared to what is required to significantly harm or kill the host, as published by the inventor in PMID=36044827). Without being bound by any theory, it is believed that the most intuitive means of understanding the profound mistakes made in sepsis research is to understand that association (mild inflammation or cytokine elevations observed in sepsis patients) has been mistaken for causation (cause of sepsis). It is well-acknowledged that association is not the same as causation. The present inventor has discovered that this false logic also exists in many current treatment approaches for sepsis. A detailed convincing discussing of these issues has been published by the present inventor in PMID=35814227 (Front. Pharmacol., “Chasing the Ghost: Hyperinflammation Does Not Cause Sepsis,” 2022 Jun. 23, vol. 13, article 910516).

Disclosed herein is a discovery by the present inventor of cause of sepsis that is heretofore unlike any other conventional theory of sepsis causation. In particular, the present inventor has discovered the entire causal chain of events leading from initial infection through molecular events that cause organ malfunction of death due to sepsis. Furthermore, by understanding the actual underlying mechanism of sepsis causality, one can also treat other clinical conditions or symptoms that are manifested by microorganism infections. Exemplary clinical conditions that can be treated using the methods disclosed herein include, but are not limited to, sepsis or severe sepsis or septic shock, heatstroke, trauma, blood loss, typhlitis, Clostridioides difficile disease, Shiga-like toxin mediated diarrhea, hemolytic-uremic syndrome (HUS), immune reconstitution inflammatory syndrome, disseminated intravascular coagulopathy (DIC), purpura fulminans, necrotizing fasciitis, hemorrhagic fevers, COVID-19, and the like. In many of these diseases, there is attendant loss of gut permeability integrity that results in translocation of pre-formed exotoxins in the gut into the circulation. Other clinical conditions that can be treated using methods disclosed herein can be readily determined by one skilled in the art having read the present disclosure.

Discussed below is a flow of events originating from initial microorganism infection up to organ malfunction or death. Unless explicitly stated or the context requires otherwise, the term “microorganism” includes bacterium, mycobacterium, fungus, protozoan (eukaryotes), virus, or other prokaryotic cells (e.g., archaea). Exemplary microorganisms infections that can be treated using compositions and methods of the disclosure include, but are not limited to, any Gram-positive coccus (aerobic or anaerobic), including Pneumococcus (Streptococcus pneumoniae), Streptococcus groups A, B, C, or G, Streptococcus anginosus group (formerly Streptococcus milleri group=Streptococcus constellatus, Streptococcus intermedius, Streptococcus anginosus), Enterococcus faecalis and Enterococcus faccium, Staphylococci (coagulase negative and coagulase positive bacteria) including methicillin-sensitive Staphylococcus aureus (MSSA), methicillin-resistant Staphylococcus aureus (MRSA), vancomycin-resistant Staphylococcus aureus (VRSA), Staphylococcus epidermidis, Staphylococcus hominis, Leuconostoc; any Gram-positive rod or bacillus (aerobic or anaerobic), including Clostridia species (C. tetani or C. botulinum, C sordellii, or C. difficile), Listeria monocytogenes, Erysepelothrix, Bacillus species (Bacillus anthracis, Bacillus cereus); any Gram-negative coccus (aerobic or anaerobic), including Neisseriae, including Neisseria gonorrhea and Neisseria meningitis, Veillonella, Bartonella species including Bartonella henselae and Bartonella quintana, Moraxella catarrhalis; any Gram-negative rod or bacillus (aerobic or anaerobic) enteric or non-enteric pathogens including Pseudomonas species, E. coli, Salmonella, Shigella, Campylobacter, Helicobacter pylori and Helicobacter cneidi, Proteus species including Proteus vulgaris and P. rettgeri, Eikenella corrodens, Aggegatibacter actinomycetemcomitans, Coxiella burnetti, Cardiobacterium hominis, Kingella kingae, Bacteroides fragilis, Serratia marcessans, Enterobacter cloacae, Citrobacter freundii, Morganella morganii, Providentia species, Haemophilus influenzae, Capnocytophaga canimorsus, Fusobacterium necrophorum and Fusobacterium nucleatum, Stenotrophomonas maltophilia, Streptobacillus moniliformis; any mycobacterium including Mycobacterium tuberculosis (TB) and non-tuberculous mycobacteria like Mycobacteria abscessus/chelonae/fortuitum/chimerae/bovis, Mycobacterium ulcerans, Mycobacterium marinum, Mycobacterium gordonae, Mycobacterium leprae; mycoplasma including Mycoplasma pneumoniae, Mycoplasma hominis; Actinomyces species including A. israelii/constellatus/naeslundii and Nocardia species; any protozoan pathogen including, Giardia lamblia, Entamoeba histolytica, Leishmania species (including L. donovani) Malaria (including species Malariae/Ovale/Falciparum/Vivax); any fungus including Candida (several species including albicans/tropicalis/glabrata/auris); any mold including Aspergillus (several species like fumigatus, flavus, niger, terreus), Mucorales, Rhizupus; dimorphic yeasts/molds including Histoplasma capsulatum, Coccidioides species (C. immitis or C. posadasii), Blastomyces dermatitidis, Paracoccidioies species; and any pathogenic virus including: Influenza viruses A, B, C, or D, Cytomegalovirus, Epstein-Bar Virus, Herpes viruses including Herpes simplex 1 and 2, Varicella-Zoster virus, Human Herpes virus 6, Severe Acute Respiratory Syndrome Coronavirus 2 (SARS-COV-2, agent that causes COVID-19), Ebola, Dengue, West Nile Virus, Respiratory syncytial virus, Parvovirus, Measles virus, BK virus, Crimean-Congo virus, Hantaviruses (including sin nombre), Human adenoviruses, JC virus, and the like.

In general, it is believed that the clinical conditions discussed herein result from exotoxins released by infecting microorganism. Unless the context requires otherwise, the terms “exotoxin,” “exotoxin associated with said microorganism,” “constitutive molecule that functions as an exotoxin,” “molecules that function as exotoxins,” “constitutive molecules with exotoxin biological activity,” and grammatical variations thereof are used interchangeably herein and refer to an exotoxin produced and released by microorganism, such as bacteria, or a constitutive molecule that functions as an exotoxin, e.g., a protein component of viruses.

In general, it is believed that all human pathogenic bacteria secrete exotoxins except for spirochetes (bacteria that cause syphilis or Lyme disease or relapsing fevers). Broadly speaking, exotoxins are substances (usually proteins) secreted by bacteria or released from bacteria following lysis/destruction of the bacterium. The function of exotoxins is generally believed to be to protect the secreting bacterium from external harm from a competing organism or to kill competing organisms. Sometimes exotoxins disable human (and other animal) host defenses that are designed to defeat pathogenic bacteria, and this includes anti-inflammatory activities. In particular, they disable/defeat/destroy host immune antipathogen substances or functions (like barriers) to further bacterial survival. An unappreciated example is that all natural antibiotics are in fact bacterial exotoxins that disable pathogen defenses (e.g., beta-lactamases) or directly disable/kill competitor pathogens (e.g., penicillin). The molecular targets that bacterial exotoxins are designed to subvert reside on or inside competing microbial competitors. This is the reason for origination. However, evolution has proceeded and conserved the same or similar molecular targets as was present in microbial competitors. For example, some bacterial exotoxins target nucleic acids or ribosomes or cell membranes or signaling molecules like mitogen-activated protein kinases (MAP kinases) or ATP, etc. Antibiotics are examples of exotoxins that do not directly target human molecules and thus can be used clinically to defeat bacteria.

It should be appreciated that methods of the disclosure are also applicable to treat animals as well as humans. In general, animals are susceptible to many of the same bacterial and viral pathogens as humans and can be treated using the methods of this disclosure. In some embodiments, the subject is a mammal. Exemplary mammals that can be treated using the methods of the disclosure include, but are not limited to, equine, porcine, canine, bovine, primate, feline, etc. Moreover, animal models have shown that exotoxins have similar effects in animals as in humans, for example, injection into animals Toxic Shock Syndrome Toxin 1 produce similar symptoms. As stated throughout herein, viral proteins possess activities that are nearly identical to those of bacterial exotoxins. These viral substances (usually proteins) can be either virus surface proteins or internal viral components. Thus, the term “the virus is the exotoxin” or “constitutive molecule that functions as an exotoxin” or “constitutive molecules with exotoxin biological activity” all include these viral proteins.

Direct actions of exotoxins may include protein synthesis inhibition or proteolytic activity that can cause epithelial/endothelia barrier malfunction. In some instances, it is believed that exotoxins is linked to induction of the “dead man's switch” that is a component of immunothrombosis via host production of vWF stands with platelet attachment. While the present disclosure provides numerous examples of bacterial exotoxins that are relevant for bacterial infections in humans, such a list is incomplete and should in no way limit the scope of the present disclosure. In fact, the scope of the disclosure includes all bacterial exotoxins and viral exotoxin-like molecules. Exemplary bacterial exotoxins and constitutive molecule that functions as an exotoxin can be divided into several classes of molecules:
a. A-B toxins: toxins that have >1 component that interacts to form a holotoxin. Usually the B component(s) form a binding site to both the targeted cell and to the A component. The A component is the active molecule that is transported into the cell by the B molecules. The A component is often an enzyme that modifies/inactivates host molecules like proteins involved in signal transduction or nucleic acids. Sometimes the exotoxin A component catalyzes an abnormal conjugation reaction inside the cell like glycosylation or addition of molecules like ADP-ribose (Clostridioides difficile binary enterotoxin). Another example is diphtheria AB toxin that catalyzes ADP-ribosylation of ribosomal EF-2 protein and halts protein synthesis.
b. Enterotoxins: refers to toxins produced by pathogenic bacteria that invade the gut. These are usually exotoxins with enzymatic activities
c. Pore-forming toxins: these exotoxins bind to the surfaces of cells and or pore that enables substances to leak into or out of the cell that disrupts cell function and can result in cell death (especially death by programmed cell death or apoptosis).
d. Cytolysins: exotoxins that disrupt and damage or kill cells as a primary function.
e. Superantigens: these exotoxins bind simultaneously to T-cells and to macrophages and force an abnormal interaction that activates both T-cells and macrophages. This results in extreme synthesis of cytokines in vitro, but this is not observed in vivo. Instead, these toxins induce massive cell death of entire families of T-cells that cripples host immune responses.

Exotoxins may be divided broadly into 3 groups depending on site of action: (a) Extracellular exotoxins act outside the cell surface. Superantigens are examples; (b) Cell-surface-acting exotoxins act on the cell surface. Examples include pore-forming toxins like pnumolysin; and (c) Intracellular exotoxins act inside cells after transportation into cells. Examples include Anthrax lethal toxin and Pseudomonas exotoxin A.

Some of examples of exotoxins and microorganism that produce them include, but are not limited to: Corynebacterium diphtheria (produces diphtheria AB toxin); Clostridium tetani (produces tetanus toxin (tetanospasmin) that causes muscle spasm (tetanus)); Clostridium botulinum (produces AB toxin and Neurotoxins A-E); Clostridium perfringens (produces alpha toxin (i.e., phospholipase C) and theta toxin (perfringolysin)); Clostridium difficile (produces C. difficile toxin A; C. difficile toxin B; and C. difficile binary toxin); Vibrio cholerae (produces cholera toxin that causes cholera); Staphylococcus aureus (produces enterotoxin in dairy foods that causes food poisoning. S. aureus also produce toxic shock syndrome toxin-1 (TSST-1) which causes toxic shock syndrome); other exotoxins produced by Staphylococci (Panton-valentine leucocidin, other leukocidins, Staphylococcal enterotoxin-A, -B, -C, -D, and -E (superantigens), Phenol-soluble modulins (cytolysins), Staphylococcal hemolysins (α, β, γ, δ), Exfoliative toxins A and B, (enzymes that disrupt cell adhesion; Streptococcus species (produces fever-causing toxins, Streptococcus toxins can also produce erythematous rash on the skin, e.g., Streptolysin-O, Streptolysin-S, DNAse); treptococcus pneumoniae (pneumococcus, Pneumolysin (a cholesterol-dependent cytolysin); Enterotoxigenic Escherichia coli (ETEC, produces exotoxins that causes traveler's diarrhea); Shigella dysenteriae (produces shiga toxins that causes diarrhea); Bacillus anthracis (produces anthrax toxin, e.g., Lethal toxin and Edema toxin, that causes anthrax); Enterococcus (Cytolysin), Listeria (Listeriolysin-O, Listeriolysin-S, Phospholipase C); Aggregatibacter (Leukotoxin A, Cytolethal distending toxin); Bordetella pertussis (Pertussis AB toxin); Campylobacter (Cytolethal distending toxin, Enterotoxin); Citrobacter (Heat stable enterotoxin, Colibactin, Shiga-like toxins, Hemolysins); Escherichia coli (E. coli, Shiga-like toxins 1 and 2, AB toxins, E. coli Secreted Protein B (EspB)); Gonococci (MafB toxins); Helicobacter pylori (CagA, VacA); Legionella (Hemolysisns, SidJ and SidH toxins); Meningococcus (MafB toxins); Morganella (Hemolysin); Providencia (Cytolethal Distending Toxin); Pseudomonas (Exotoxin A, Pyocyanin); Salmonella (Typhoid toxin, SpvB, ArtAB, SboC/SepC); Serratia (ShlA); Shigella (Shiga toxins); Vibrio cholera (Cholera AB toxin (increased intracellular ADP by ADP-ribosylating intracellular G-proteins)); Vibrio (RtxAl toxin in Vibrio vulnificus); Yersinia (YOP proteins (YOPE/M/O/PH/t.); Mycoplasma (Community-Acquired Respiratory Distress Syndrome Toxin (CARDS toxin)); ycobacterium tuberculosis (Early Secreted Antigenic Target 6 kDa (ESAT-6), Culture Filtrate Protein 10 kDa (CFP-10), Tuberculous necrotizing toxin *TNT); Mycobacterium ulcerans (Mycolactone); Candia (Candidalysin), etc.

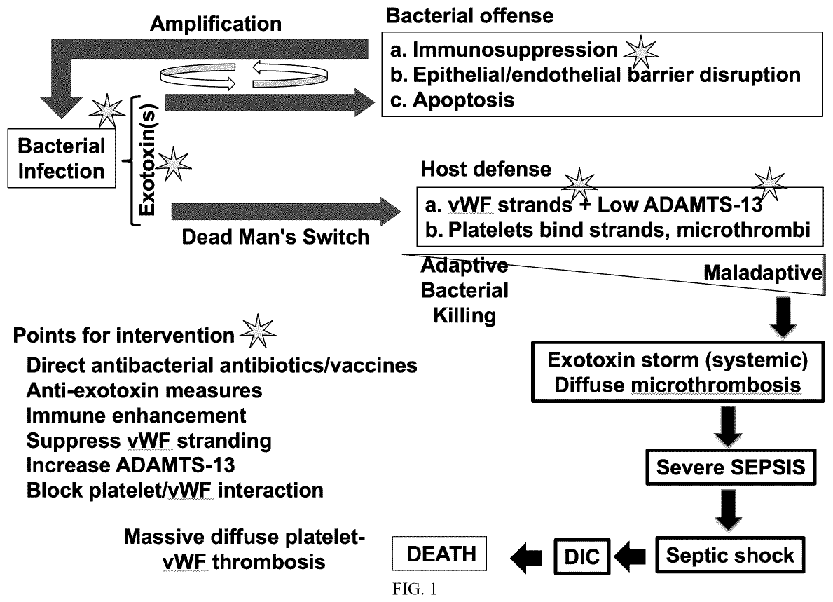

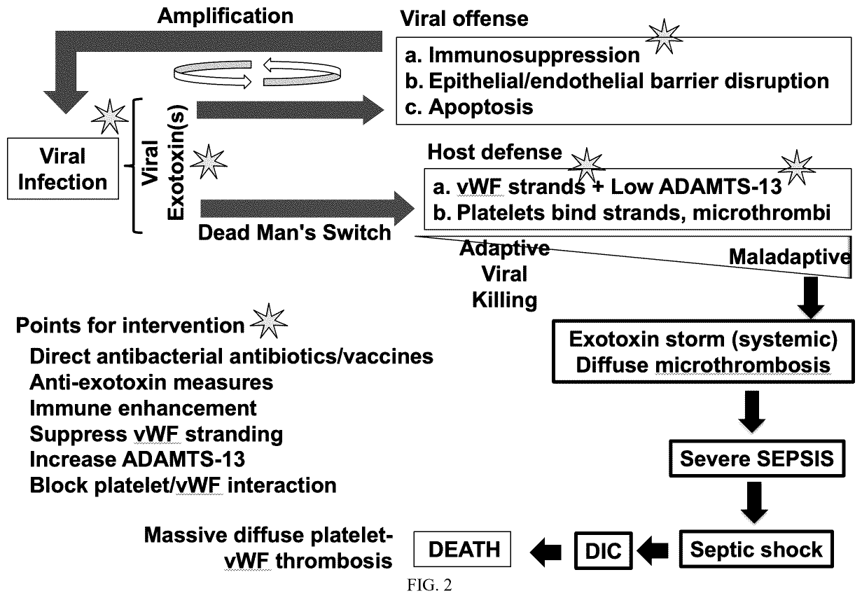

As illustrated in FIGS. 1 and 2, exotoxins suppress or subvert host's anti-pathogen innate immunity, and therefore, facilitates pathogen survival within the host. Unless the context requires otherwise, the terms “host,” “subject,” and “patient” are used interchangeably herein and refer to mammals, such as primate, canine, feline, bovine, porcine, or equine. In some embodiments, the host is human. The term “exotoxin” refers to a functional definition that may manifest differently in different microorganisms. For example, for bacteria and fungi, exotoxins are products (e.g., proteins) that are produced and secreted by bacteria and fungi, respectively. For viruses, exotoxin refers to proteins that are part of the virus itself, such as the envelop protein of the virus (or portion(s) thereof) or internal viral proteins. Thus, the term “exotoxin” refers to any protein(s) that are released, produced, or resides on or within the microorganism that elicits the detrimental biological effects on the host that depress the host immune response and can eventuate in sepsis or a sepsis-like clinical condition.

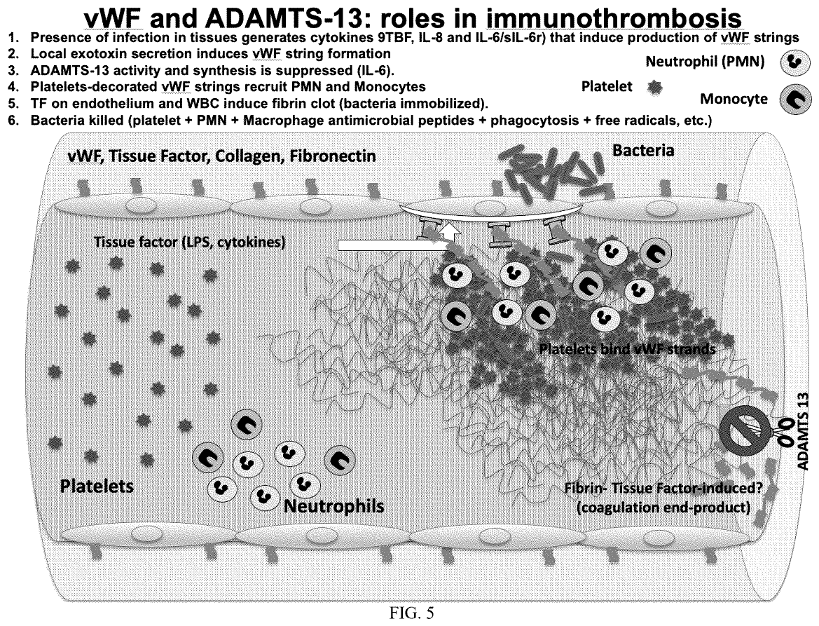

Referring to FIG. 1, bacterial exotoxins are synthesized and secreted with the function of suppressing host immunity to evade killing by the host. This is shown in the top of the figure labeled “Bacterial offense”. Exotoxins cause immunosuppression and epithelial barrier disruption and apoptosis (programmed cell death). These activities all contribute to degrade host capacity to defeat the pathogen. The host has developed a clever defense against bacteria by entering the Dead Man's Switch physiology when cells are attacked by bacterial exotoxins. Endothelial cells (cells that line blood vessels) respond to exotoxin presence by initiating a series of actions that eliminate bacteria. Exotoxin-induced reduction in protein synthesis in the endothelial cell quickly induces production of long vWF strands on the endothelial cell surface that traps pathogens in a sticky mass and attracts immune cells to the region of infection for the purpose of killing the pathogen (see discussion of FIG. 5 below for detailed description of adaptive immunothrombosis). The arrows and small circular arrows shown at the top of FIG. 1 show that poor control of the pathogen by the host can result in a vicious cycle of continued immunosuppression that permits further replication of the pathogen and production of more exotoxin that causes yet more immunosuppression. This enables survival and replication of the bacterium with further synthesis of exotoxins. Exotoxins made in large amounts can produce pathological blood levels and exotoxins disseminate widely throughout the body. This results in counterproductive diffuse activation of the Dead Man's Switch mechanism that is so diffuse as to be counterproductive. This results in creation of widespread microthomboses that leads to organ hypoperfusion and organ malfunction or death. This is depicted by the increasing size of the wedge going left to right below the description of the Dead Man's Switch (from “Adaptive Bacterial Killing” to “Maladaptive”). Left side shows the adaptive activity of this Switch in killing bacteria when the Dead Man's Switch is deployed locally at the site of exotoxin presence. Right side shows maladaptive diffuse activation of the Dead Man's Switch caused by disseminated exotoxin in blood (referred to as “exotoxin storm”). In this case there is diffuse systemic formation of microthrombi that chokes off blood supply to organs and can result in organ malfunction or even death. The Points for Intervention indicate locations for therapeutic intervention discussed in this application. As discussed infra vWF refers to von Willebrand Factor, ADAMTS-13 refers to A Disintegrin and Metalloprotease with a Thrombospondin-1 Motif member 13, and DIC refers to Disseminated Intravascular Coagulopathy DIC.

Referring to FIG. 2, viral constitutive component proteins that reside on the viral surface or sequestered within the viral capsid itself possess exotoxin activities similar to those of bacterial exotoxins (e.g., components of viral particles or the virus itself is the exotoxin). These viral exotoxins then produce sepsis disease in the same fashion as described for bacteria in FIG. 1.

The host has evolved a highly specific mechanism to counteract the detrimental effect of exotoxins. The adaptive mechanism of host can be thought of as a nature's “dead man's switch” or “dead man's device,” which is a well-known design feature people have built into some devices to enhance safety. A detailed description of dead man's switch is provided below. Mammalian (i.e., a host such as a primate, canine, feline, bovine, porcine, equine, etc.) cells have evolved in a way to detect the presence of microorganism exotoxins (or constitutive viral proteins that function as exotoxins) and respond by activating the “dead man's switch” mechanism within endothelial cells that line the walls of blood vessels. This mechanism usually succeeds in eliminating the offending pathogens. However, if pathogen replicates to a critical amount such that the amounts of exotoxin(s) overwhelm the capacity of host defenses to contain the pathogen, the exotoxins produced by the pathogen can increase in amounts in blood and surpass critical levels. The exotoxin(s) then disseminate widely throughout the body via the bloodstream and activate the “dead man's switch” device on a diffuse widespread scale systemically. This can eventuate in organ malfunction or death of the infected host.

A “dead man's switch” or “dead man's device” is an engineering term that refers to a design feature intentionally incorporated into devices or machines that lowers the risk of injury to people if the device fails or is mishandled (see Wikipedia entry at: https://en.wikipedia.org/wiki/Dead_man%27s_switch). Machines are designed in a way that ensures that in case of machine failure or accident, the machine enters a “safe mode” that minimizes harm. A classic example is the design of commuter trains, where a constant application of pressure against a control accelerator handle (“switch”) is required for the train to move under power. If the accelerator handle is released it returns under spring tension to the resting position and the train will slow down and stop. This feature is intentionally built into the train design in order to discontinue motion under power in the event the driver/engineer has a medical emergency (example, heart attack) or leaves his/her station for some reason. In this way, a failure in the device (train controller disabled or otherwise indisposed), the device (train) shuts down to avoid continued movement without the supervision of the operator. Another common example is home handheld power tools that require constant finger pressure on the tool switch (often a trigger) in order to maintain operation. If the tool (example, power drill) is dropped, the drilling motion stops almost immediately and thereby reduces the risk for damage to the operator and surroundings (especially the operator's foot or the floor).

Interestingly, heretofore unknown, the present inventor has discovered that the host response to infections appears to embody a dead man's switch device to detect and combat pathogen infections. Following infections that cause “malfunction” of the cell (especially endothelial cells that line all blood vessels), the cell enters a mode of operation designed to eliminate the pathogen causing the malfunction (a “safe mode”). This host safe response comprises several coordinated activities within endothelial cells that line blood vessels. Since all organs require a blood supply, the dead man's device is ubiquitous throughout the body. In circumstances of non-systemic non-severe infections, the dead man's device mechanism is adaptive and promotes survival of the host.

The dead man's device operates in endothelial cells that line all blood vessels in the body. Pathogenic bacteria and viruses possess one and only one means to induce pathology in hosts, namely, exotoxins in the case of bacteria and exotoxin protein components for viruses (“the virus itself or is the exotoxin” see above). Exotoxins comprise the only offensive capability of pathogens. This contrasts sharply with the current accepted pathogenesis of sepsis which comprises excessive inflammation, i.e., cytokine storm, in the host as the mechanism for inducing pathology in the host. Since the treatment approach to sepsis that has used myriad attempts to block inflammation have failed (see PMID=35814227, Front. Pharmacol., “Chasing the Ghost: Hyperinflammation Does Not Cause Sepsis,” 2022 Jun. 23, vol. 13, article 910516), it is rational to focus on a sepsis cause that emphasizes a role for the pathogen (e.g., infecting bacterium, virus, fungus, or protozoan) instead of the response of the infected host.

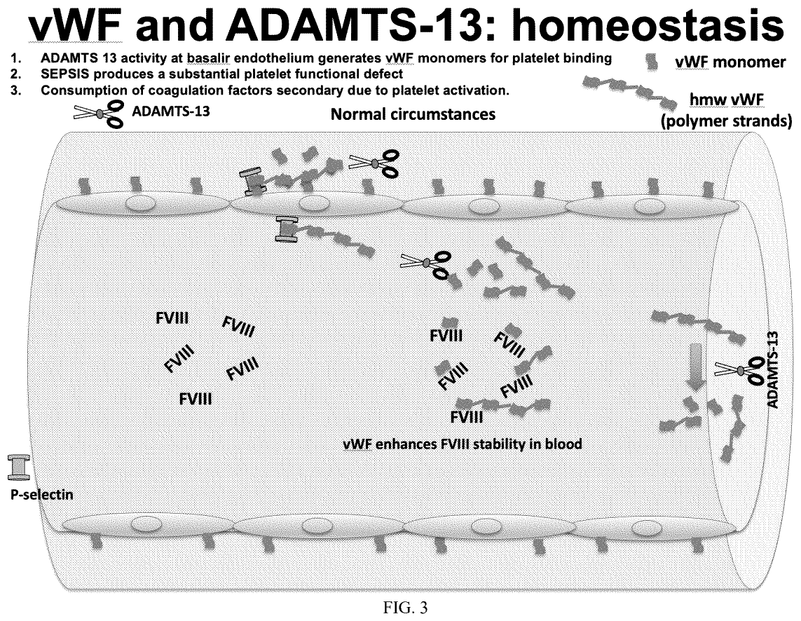

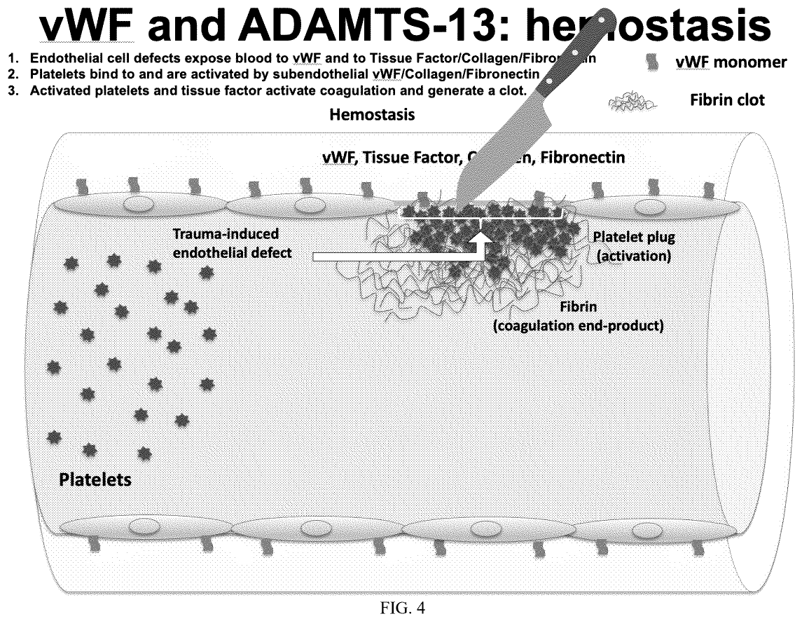

To date, no one has recognized pathogenic microorganisms themselves cause sepsis. As disclosed herein, once one focuses on the pathogens as the cause of sepsis, logic leads to the inexorable conclusion that only pathogen offensive capability (exotoxins secreted from bacteria or as components of viral particles) can induce organ malfunction or death in hosts. When exotoxins contact or enter blood vessel endothelial cells, they alter or subvert cell function in a way that damages or kills the endothelial cell or reduces its capacity to resist bacterial or viral replication within the cell. A common end-stage effect of almost all exotoxin activities on endothelial cells involves protein synthesis inhibition or cessation (especially if the cell is destined to be killed by the toxin). Of paramount relevance for understanding this unique insight into sepsis pathogenesis is the normal physiological role of von Willebrand factor or vWF. As illustrated in FIGS. 3 and 4, vWF is a hemostasis molecule that is “sticky” for platelets and binds to them tenaciously (an analogy would be to compare platelet attraction to vWF like flies to flypaper).

Referring to FIG. 3, the normal biological functions of vWF molecules are shown. Briefly, vWF is produced inside endothelial cells as large vWF strands that are composed of many vWF monomers attached to each other like beads on a string. There is a larger storage pool of these vWF strings inside these cells. The vWF long strands are secreted to the cell surface where they attach to cell surface P-selectin, which anchors the vWF strands to the surfaces of the endothelial cells. The large vWF strands now resemble streamers that are waving in the “breeze” of the flowing bloodstream. The vWF strands are the sliced into smaller multimers and also detached from the P-selectin through the action of ADAMTS-13 metalloprotease. ADAMTS-13 is NOT stored inside the cells but is secreted as soon as the molecule is synthesized. The freed vWF monomers and polymers then bind to the blood coagulation Factor VIII. In addition to long vWF strands secreted onto the endothelial surface that contacts the blood flow, vWF is also secreted from the endothelial cells into the tissues beneath the endothelium (opposite side of blood flow) where these molecules take up residence in the surrounding tissues.

As shown in FIG. 4, in one of their normal physiological roles, vWF participate in hemostasis, or stoppage of bleeding after an injury. In FIG. 4, an injury or cut is represented by a knife. This damages the endothelial cells and blood then contacts the tissues that reside beneath the endothelial cells. vWF molecules that have been previously secreted into the sub-endothelial tissues (see FIG. 3) tightly bind platelets due to molecular interaction and this forms a platelet plug that stops further bleeding (similar to a bandage). There is also subsequent activation of the blood-borne clotting cascade proteins that bolsters the platelet plug by forming a fibrin clot. Thus, when the host sustains an injury (FIG. 4), there is often bleeding into the tissues where vWF normally is present. Since vWF attracts and binds platelets, a mass of adhered platelets forms a platelet plug that represents the first adaptive response to a mechanical defect that stops further bleeding and seals the wound. This is usually followed by activation of the clotting cascade system and results in clot formation that stops bleeding and initiates the complex process of tissue repair. The unusual biology of vWF production appears to serve 2 different purposes. The first is facilitation of hemostasis or clotting to limit blood loss and repair damage. The second purpose is to serve as a component of innate immunity as a means to fight infections. This duality of function makes biological sense since any region of damage that results in bleeding can also serve a portal or entry for pathogenic bacteria or viruses Thus, combining roles for hemostasis and immunity serves 2 related purposes. The biology of vWF is unique in the body and its unusual nature appears to derive from its role in the “dead man's switch” biology designed to combat pathogens. Normally endothelial cells secrete vWF molecules constitutively in the form of long strands composed of many vWF molecules joined end to end like beads on a string., Endothelial cells possess substantial amounts of pre-synthesized vWF strands or long strings sequestered inside endothelial cells within Weibel Palade Bodies (WPB).

The unusual and unique natural course of events for vWF production within the body is outlined below. Giant strands of vWF polymers (“beads on a string”) are secreted from the endothelial cell interior and loaded onto the endothelial cell surface to form giant vWF strands (see PMID=24448155 (Stockschlaeder, M. et al., Blood Coagulation & Fibrinolysis, “Update on von Willebrand factor multimers focus on high-molecular-weight multimers and their role in hemostasis”, 2014, 25(3), pp. 206-216, April 2014)). P-selectin is the molecule on the surface of endothelial cells that binds and anchors the long vWF strands to the endothelial cell surface). The vWF strands (made of many vWF monomers strung together repeatedly to form the strands) are sliced into monomers and small polymers by the ADAMTS-13 metalloprotease. Infections are associated with production of exotoxins and results in disrupted/suppressed/stopped protein synthesis of the endothelial cell. In this case, one of the first molecules that decreases in levels is ADAMTS-13. This is so because there is no storage pool for this molecule inside cells, and it is normally secreted as soon as it is synthesized. Therefore, ADAMTS-13 action diminishes immediately after protein synthesis disruption. Under conditions of exotoxin attack on the endothelial cell, large vWF strands continue to decorate the external surface of endothelial cells since this is a constitutive process and there is a large storage pool of vWF within Weibel Palade bodies that is unaffected by diminished protein synthesis. Moreover, there is increased secretion and appearance of large vWF strings following exposure of endothelial cells to bacterial exotoxins or by infection with viruses (including the COVID-19 virus). See, for example, PMID=22990960 (Freitas, C. et al., Mem Inst Oswaldo Cruz., “The infection of microvascular endothelial cells with ExoU-producing Pseudomonas aeruginosa triggers the release of von Willebrand factor and platelet adhesion,” 2012, 107 (6), pp. 728-34); and PMID=20644116 (Huang, J. et al., Blood, “Shiga toxin B subunits induce VWF secretion by human endothelial cells and thrombotic microangiopathy in ADAMTS13-deficient mice,” 2010, 116(18), pp. 3653-9.). The large vWF strands then recruit platelets and immunocytes (macrophages, lymphocytes, and polymorphonuclear neutrophils (PMN) to the strand surfaces (strands act somewhat like flypaper (vWF normal function) for these immunocytes). There is activation of the coagulation cascade forming fibrin clot material that can trap pathogens near the endothelial cell surface and thus prevent pathogen dissemination via the circulation (immunothrombosis, see FIG. 5, as described in PMID=36835934 (Marcos-Jubilar, M. et al., J Clin Med., “Immunothrombosis: Molecular Aspects and New Therapeutic Perspectives,” 2023, 12(4), 1399)).

Briefly, FIG. 5 depicts the host-evolved Dead Man's Switch or Device that is designed to identify, locate and eliminate pathogens. The presence of exotoxins or exotoxin-functioning molecules damage or kill cells. Protein synthesis is an invariant consequence of the presence of significant amounts of exotoxins. Since ADAMTS-13 is not stored but only synthesized constitutively, production of ADAMTS-13 ceases immediately after protein synthesis is inhibited. At the same time, secretion of vWF long strands continues and these are anchored to the surface of endothelial cells by binding to surface P-selectin. These attached vWF long strands then bind platelets passing by in the blood, and there is secondary binding of immunocytes (monocytes, lymphocytes, and neutrophils) to these platelet-encrusted vWF strands. This is an adaptive response when there is a focus of infection with localized presence of exotoxins. The attracted immunocytes (platelets, monocytes, lymphocytes, and neutrophils) will identify the pathogen by engagement of pattern-recognition receptors (like TLRs) and then kill the invading pathogen Host cell killing mechanisms can include phagocytosis and digestion, production of reactive oxygen species, or secretion of lytic enzymes. Also, there is activation of the clotting cascade by the vWF-platelet microthrombi, and this serves to trap and immobilize pathogens and blocks dissemination. During the usual course of events there is localized infection and thus the disruption of endothelial cell functioning caused by exotoxins is an accurate locator of the presence of the infecting pathogen. These events overall initiate a Dead Man's Switch or Dead Man's Device since attack against the endothelial cell causes the cell to fail or die. This initiates a series of events (block of ADAMTS-13 in the local environment and establishment of attached long vWF strands) that adaptively eliminates the pathogen. The net result of activation of the Dead Man's Switch is initiation, focusing, and amplification of innate immune power at the site of infection; where it is needed.

The above-described events represent an anti-pathogen response that activates several host-favorable activities of the innate immune response. For example, the formation of vWF strands decorated with platelets activates the coagulation cascade with formation of sticky adhesive clot material around the strands. This serves to trap and immobilize pathogenic bacteria and viruses. This is a well-characterized role for clotting in the body and is known as IMMUNOTHROMBOSIS (PMID=36835934 (Marcos-Jubilar, M. et al., J Clin Med., “Immunothrombosis: Molecular Aspects and New Therapeutic Perspectives,” 2023, 12(4), 1399)). This mechanism is designed as a localized response that occurs in the physical presence of bacterial or viral pathogens. Bacterial exotoxins in most situations are secreted into the local microenvironment and thus act at nearby endothelial cells. Similarly, viral exotoxin-like proteins act at the site of viral presence. Therefore, events described above serve to identify the location of pathogen activity and to focus and amplify pathogen-combatting power at the site of pathogen presence. Strand formation on the endothelial cells recruit and activate immunocytes (platelets, lymphocytes, PMN and monocytes) at the site of pathology/infection and these cells can focus immune power at the site of infection. The result is focused and amplified antipathogen activity at the site of infection where increased immune power is needed (indicated by the initiating activity of exotoxin molecules). The usual outcome is pathogen elimination.

Considerations described above instantiate a dead man's switch or device. Endothelial cells damaged by exposure to bacterial exotoxins or viral exotoxin-like proteins represents a failure of cell function and indication of local danger. This causes diminution of protein synthesis (including reduced synthesis of vWF strand-cleaving ADAMTS 13) and increased expression of vWF strands. The strands trap and localize pathogens and prevent their spread throughout the body, and the strands recruit/focus immune cell activities at the site of infection. The net result is enhanced pathogen killing induced by endothelial cells under stress. Therefore, pathogen-induced endothelial cell failure causes the endothelial cell to enter an adaptive series of events (routine) that counteracts or neutralizes the offending cause. Since the source of the offending bacterial exotoxin(s) or viral exotoxin-functioning proteins is usually near the affected endothelial cell, the localized multi-pronged anti-pathogen response is adaptive and likely to immobilize, inactivate, and kill the offending pathogenic microorganism. This is a quintessential example of a dead man's device or switch, where stressed cells enter a subroutine or mode that mitigates overall damage to the host (infected person).

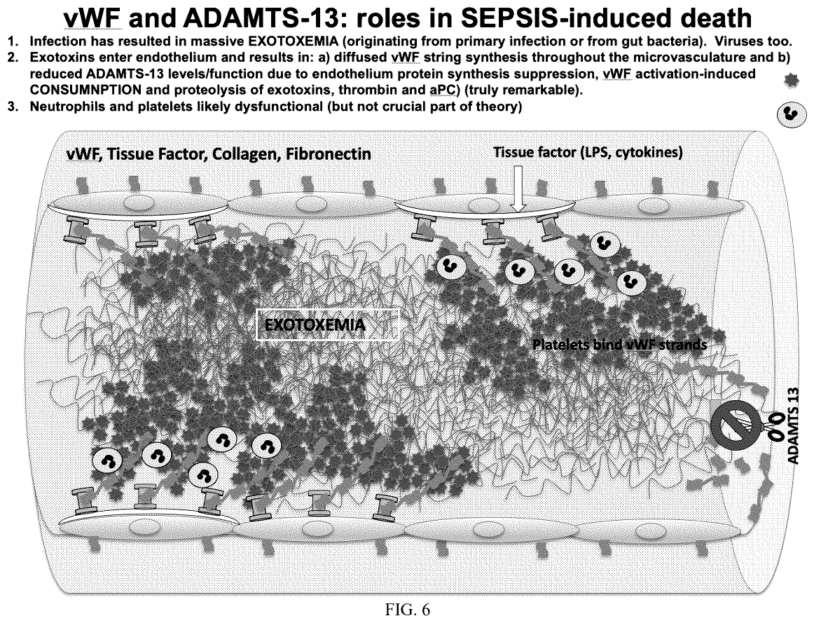

As described above, the design of the endothelial dead man's switch generates large vWF strands on the vascular surface that contacts the circulation in response to exotoxins or viral particles with exotoxin function. This mechanism is believed to be a product of host evolution and is adaptive for the purpose of pathogen elimination. However, this mechanism can become maladaptive in the case of overwhelming systemic amounts of bacterial exotoxins or viral replication. When pathogens gain the upper hand in the battle against the host, there is prodigious production of bacterial exotoxins or extensive replication of virus and concomitant increase in viral proteins with exotoxin function. This results in diffuse systemic runaway activation of the dead man's switch which then can result in a vicious cycle as illustrated in FIG. 6.

Referring to FIG. 6, as described for FIG. 5, activation of the Dead Man's Switch in the presence of a localized infection is adaptive for the host. However, since exotoxins (or exotoxin-functioning molecules in viruses) are inhibitors of the immune response, sometimes the pathogen overwhelms the innate immune system of the host and this establishes conditions that permit rapid and uncontrolled pathogen replication and synthesis of more and more exotoxin(s). If unchecked, exotoxin levels in blood become critical and activate the Dead Man's Switch mechanism diffusely throughout the body. In such cases there is formation of microthrombi throughout the body and this causes downstream tissue ischemia that causes organ malfunction or even death. This is the established cause of death in thrombotic thrombocytopenia purpura (TTP). Notice that dissemination of large concentrations of exotoxins in blood disables the ability of the Dead Man's Switch mechanism to localize the offending pathogen. Exotoxin is now everywhere, and a specific localized focus Dead Man's Device activation cannot isolate and eliminate pathogens.

Bacterial exotoxins and exotoxin-functioning viral proteins are designed to degrade innate immune function in the host and in this way promote pathogen survival and replication. These same exotoxins and viral proteins are detected by vascular endothelium and this activates the endothelial dead man's switch device designed to neutralize the pathogens. In most cases this results in elimination of the pathogen. In some cases, the pathogen(s) get the upper hand and profoundly suppress host innate immune function (the primary activity of exotoxin proteins is to disable immune function to facilitate survival of the infecting microbe) which permits massive pathogen proliferation and production of large amounts of exotoxins or like-functioning viral proteins. A vicious cycle is established where exotoxin-induced immune suppression permits pathogen replication and further enhanced exotoxin synthesis and more and more endothelial surface vWF standing that occurs diffusely throughout the body. Since exotoxins all function to suppress innate immunity, exotoxins are anti-inflmmatory. Therefore, traditional approaches to treating sepsis using anti-inflammation measures (see TABLE 1) amount to “aiding and abetting” sepsis rather than measures that can cure/treat sepsis.

This forward-feeding vicious cycle is observed empirically, with blood concentration of bacterial exotoxins documented as high as 35,000,000 μg/mL in infected rabbits (PMID=24684968 (Liu et al., Trends Microbiol., “Anthrax lethal and edema toxins in anthrax pathogenesis,” 2014, (6), pp. 317-25)) in cases of anthrax bacterial infections, and with viral loads (viral particles per mL blood) reaching levels >1 billion viral copies per mL in cases of Dengue sepsis (PMID=16983615 (Wang, W. et al., Clin Infect Dis., “Slower rates of clearance of viral load and virus-containing immune complexes in patients with dengue hemorrhagic fever,” 2006, 43(8), pp. 1023-30)). In these cases, there is diffuse activation of the dead man's switch mechanism within endothelial cells throughout the body. This is not the situation that human evolution intended. The dead man's switch mechanism is useful/adaptive in the presence of localized infection. It becomes maladaptive in cases of diffuse/overwhelming infections when the dead man's devices is activated diffusely This results in formation of microthromboses (sometimes referred to as thrombotic microangiopathy) in small blood vessels and capillaries composed of vWF strands+platelets+clotting factors. This results in diffuse compromise of microvascular blood flow and necrosis or damage of cells downstream from the microthrombi. Moreover, when exotoxin production becomes excessive, exotoxins disseminate throughout the body via the bloodstream. In this circumstance, the presence of exotoxin can no longer be used as an indicator of a nearby pathogen.

There is an explanatory triad of phenomena well-known in the clinical and scientific literature that can be used to corroborate the sepsis pathogenesis mechanism described above. This triad provides proof that the dead man's mechanism fully accounts for mortality, shows that infectious diseases can cause this mortality, and shows clear evidence that the dead man's device is activated diffusely during severe stages of sepsis. The triad consists of one-thrombotic thrombocytopenia purpura (TTP), two-Hemolytic uremic Syndrome (HUS), and three-disseminated intravascular coagulation (DIC) that together provide rationale for the sepsis pathogenesis described above.

TTP is a disease caused by genetic or acquired functional deficit of ADAMTS-13. Briefly, ADAMTS-13 is the metalloprotease that cuts vWF polymers into monomers and oligomers and thus frees vWF to enter the circulation and detaches long vWF strands from surfaces of endothelial cells (recall that P-selectin on the surfaces of endothelial cells anchors long vWF strands to the surfaces on endothelial cells and the vWF strands thus “wave in the breeze” of blood flow). Clinical manifestations of TTP are caused by microthrombi composed of vWF and adherent platelets and small amounts of adherent clot. One of the key features or observations of TTP for this disclosure is the mortality due to TTP was approximately 90% before the introduction of plasma administration therapy. Intravenous infusion of plasma is an approved therapy because plasma contains fresh functioning ADAMTS-13 that is added to the blood of persons with TTP-associated ADAMTS-13 deficiency. It is believed that vWF strands adorned with adherent platelets and clot are a sufficient explanation for TTP mortality. In other words, the near 100% fatality caused by deficiency of ADAMTS 13 and attendant vWF stranding establishes that the similar phenomenon occurring in sepsis can indeed account for sepsis mortality and organ malfunction. As described, there is remarkable deficiency of ADAMTS-13 in sepsis see for example: https://epag.springeropen.com/articles/10.1186/s43054-023-00219-1 #). Therefore, the mechanism disclosed herein accounts for sepsis organ malfunction or death.

Hemolytic-Uremic Syndrome or HUS is a disease caused by infection of the GI tract with certain strains of Escherichia coli (E coli) bacteria. HUS is characterized by a microangiopathy that is very similar to that noted in cases of TTP and sepsis. It is accepted that E coli production of a family of exotoxins known as “Shiga-Like toxins” or SLTs is the cause of this non-sepsis disease. In fact, it has been shown experimentally that exposure of endothelial cells to SLTs results in appearance of large vWF strands atop endothelial cells (PMID=21816831 (Liu F et al., Blood, “Shiga toxin (Stx) 1B and Stx2B induce von Willebrand factor secretion from human umbilical vein endothelial cells through different signaling pathways,” 2011, 118(12), pp. 3392-3398)). The importance of HUS for enablement of this application is the link it forges between the high mortality of TTP and bioactivity of bacterial exotoxins. TTP has a near 100% fatality without treatment. This unequivocally links the mortality of TTP to the biological activity of exotoxins. It is thus established that exotoxins possess bioactivities that result in molecular events nearly identical to those observed in TTP. Since TTP has near 100% mortality, exotoxins can indeed function as proximate causes of sepsis.

Sepsis that is severe enough to cause organ malfunction or death often enters a clinical state known as disseminated intravascular coagulopathy or DIC. This clinical syndrome is characterized by diffuse and systemic activation of the clotting cascade and slight to profound diminution of platelets in blood. It is associated with very high mortality of approximately 45-78%. Detailed analysis reveals DIC is a manifestation of the thrombotic microangiopathy of the sort predicted by this disclosed theory that follows severe infection complicated by severe sepsis with organ malfunction or death. A prototype extreme example of DIC in sepsis is in the disease called “purpura fulminans” that can arise in patients (usually children) infected with Neisseria Meningitis. This specific sepsis variant is a well-described example of sepsis-induced microangiopathy thought to account for disease (PMID=29157918 (Colling, M. et al., Transfus Med Rev., “Purpura Fulminans: Mechanism and Management of Dysregulated Hemostasis,” 2018, 32(2), pp. 69-76)). This sepsis theory states there is a single universal biological process in sepsis with varying degrees of severity but is uniformly present in severe cases. It is always (or nearly always) the cause of organ malfunction or death. Clinical and laboratory observations in DIC establish a link between high mortality (TTP), exotoxin causality (HUS), and clinical manifestation in sepsis (DIC).

The word “coagulation” occurs in DIC and there is consistent evidence for activation of the coagulation clotting factors cascade in sepsis. It is reported that sometimes clotting is activated to the point that a “consumptive coagulopathy” or extreme activation and exhaustion of coagulation components sufficient to cause sepsis pathology. This idea is often invoked as the cause of disease in viral infections known as “hemorrhagic fevers” like severe infections with Ebola virus. However, no clinical evidence shows coagulopathy is clinically excessive, and excessive bleeding has never been shown to be the cause of sepsis pathology in any viral hemorrhagic fever (including Ebola). Not one case-report has demonstrated this mechanism for sepsis disease. Moreover, numerous attempts to arrest the consumption of coagulation molecules have failed in clinical trials in sepsis patients including use of heparin, warfarin, activated Protein C (XIgris™), thrombomodulin, recombinant antithrombin-3, tissue plasminogen activator, and tissue-factor pathway inhibitor (see TABLE 1 and PMID=35814227). Therefore, the rationale supporting coagulation-pathway inhibitors to treat sepsis is confused and incorrect. There is indeed activation of coagulation during sepsis, but it does not contribute substantially to sepsis pathogenesis. It is not clinically significant. Activation of clotting during sepsis is epiphenomenal (a non-causal association) and unrelated to organ malfunction or death; it is an association that is not causal. This small amount of clot is not clinically significant in sepsis pathogenesis, as established by inability to treat sepsis with anti-clotting drugs. As discovered by the present inventor, the true pathogenesis of sepsis involves vWF stranding with associated platelet mass that produces the pathology. The purported association between coagulopathy and sepsis is an example of a recurring theme of misdirected thought in sepsis pathogenesis. Association is confused for causation. The presence of increased (but sub-lethal) cytokines in sepsis is another example of this faulty association, and there are many other examples of this kind of conceptual error. These misdirections in the current understanding of sepsis again highlight the radically novel approach reported in this discovery disclosure.

While there is a small increase in inflammation during natural sepsis, this small elevation represents the host response to infection that is designed to kill the invading pathogen, i.e., microorganism. However, it has been observed that in severe cases of sepsis this amount of inflammation is insufficient to kill the microorganism that may cause sepsis. The reason host inflammation is insufficient is the microbe possesses mechanisms to actively suppress or blunt the host innate immune response to infection through the activity of exotoxins, as described above. Moreover, the small increase in inflammation during natural sepsis never sufficient to damage the host. In fact, the inventor published the only known detailed analysis of inflammatory cytokine levels in sepsis. This analysis proved beyond doubt levels of these molecules are several orders of magnitude lower than those capable of producing serious harm in patients. (PMID=36044827 (Gharamti et al., Cytokine, “Proinflammatory cytokines levels in sepsis and healthy volunteers, and tumor necrosis factor-alpha associated sepsis mortality: A systematic review and meta-analysis,” 2022, 158, 156006)). It is believed that the host tries to generate sufficient immunity or inflammation to kill the pathogen, whereas the pathogen tries to suppress immunity or inflammation in an attempt to promote pathogen survival. Viewed in this light, it is profoundly misdirected to deploy inflammation-suppressing measures to treat sepsis. This explains the exceptionless failure of this approach to sepsis treatment (TABLE 1) and again underscores the novelty of this application.

Based on this discovery by the present inventor, some aspects of the disclosure provide methods for treating sepsis or a sepsis-like clinical conditions. Other aspects of the disclosure provide methods for preventing development or occurrence of sepsis or sepsis-like clinical conditions. Still other aspects of the disclosure provide methods for producing a therapeutic agent that is capable of treating, preventing, or reducing the severity of sepsis or sepsis-like clinical conditions. Most of the methods and compositions disclosure herein relate to preventing, inhibiting, reducing, or blocking the actions of exotoxins of microorganism origin that infects the host.

In some cases bacteria that invade the body are sufficient by themselves to generate large amounts of exotoxins that can enter the bloodstream and circulate throughout the body and cause sepsis. One example is anthrax lethal toxin. After anthrax spores germinate (convert into replicating bacteria), they invade tissues and bloodstream and can replicate in blood to levels of about 100 million bacteria per mL blood; this is a sufficient bacterial load within the blood to produce amounts of exotoxin required to cause sepsis. Up to 35,000,000 μg/mL anthrax exotoxin has been measures in a rabbit model of anthrax-induced sepsis (PMID=24684968).

However, in many cases of sepsis, the inciting (i.e., original or initial) infection may not be associated with sufficient numbers of bacteria in blood or in tissues to produce enough exotoxins to cause sepsis. In some instances, there is evidence that epithelial barrier dysfunction plays a role during sepsis. Since the gut contents are normally largely excluded from the bloodstream by an intact epithelial gut barrier, increased gut permeability may be caused by exotoxins produced by the inciting infection, or by bacterial surface components that include endotoxin (e.g., a cell-surface component of Gram-negative bacteria and very different from an exotoxin) or lipoteichoic acid (cell surface component of Gram-positive bacteria). Without being bound by any theory, in some cases it is believed that both exotoxins and bacterial surface components function together to cause gut hyperpermeability. Since the colon alone contains about 40 trillion bacteria as endogenous flora at all times, this is likely a contributor of systemic exotoxin due to exotoxin translocation across the gut barrier that enters the bloodstream and produces a tremendous increase in circulating exotoxin amounts that (along with exotoxin from the inciting bacterial infection) produce immunosuppression and (at higher concentrations) additional epithelial/endothelial barrier dysfunction plus apoptosis. This discovery by the present inventor is supported by evidence showing selective decontamination of the digestive tract with antibiotics significantly lowers sepsis mortality, and about 50 million picogram (pg) of total exotoxins have been measured inside the colon during gastrointestinal infections (Clostridium difficile, PMID=29788296).

Other aspects of the disclosure are directed to a method for treating sepsis by targeting exotoxins rather than using antibiotics to target the infecting bacteria themselves. That is, different from conventional goal of selective decontamination of the digestive tract with antibiotics as a sepsis treatment, which is designed to reduce inflammation caused by bacteria inside the gut that translocate into blood during severe infection and then causes systemic inflammation. Some methods of the disclosure target exotoxins specifically. In other embodiments, methods of the disclosure target exotoxins in addition to using antibiotics to kill the bacteria that are producing exotoxins.

Fungal sepsis: Although not as common as bacterial sepsis, fungal infections can also cause sepsis, and severe sepsis results in organ malfunction and even death. The mechanism of fungal sepsis is similar to that of bacterial sepsis as discussed herein. During a serious fungal infection, there is likely fungal secretion of exotoxins that cause immunosuppression and endothelial/epithelial barrier disruption and apoptosis at higher concentrations. Although few empirical data are available for fungal sepsis, the present inventor believes the pathogenesis pathway is the same as for bacterial and for viral infections with nearly indistinguishable clinical outcomes. Early work has identified a cytotoxic exotoxin produced by Candida species called candidalysins (PMID=35073742 (Richardson et al., mBio., “Candidalysins Are a New Family of Cytolytic Fungal Peptide Toxins,” 2022, 13 (1), c0351021)). Exotoxin bioactivity is the sole cause of sepsis initiation according to this disclosure.

Accordingly, some aspects of the disclosure provide fungal sepsis treatment by targeting the fungal exotoxin(s), the subject's own gut bacteria, exotoxins produced by the subject's own gut bacteria, blocking components of the Dead Man's Switch. (see sections—41-004) or a combination thereof. In some embodiments, one or more of these methods are used in conjunction with direct acting antifungal drugs.

Viral sepsis: Viruses are well-described causes of sepsis. Examples of viral sepsis include viral hemorrhagic fevers like Ebola, Marburg, Dengue disease, as well as other viral infections like Chikungunya and Hantavirus infections (causes of Hantavirus pulmonary syndromes or Hantavirus Renal syndromes). Viral causes of sepsis share the interesting characteristic that they produce very large viral loads in the circulation. In some instances, during viral sepsis, virus blood concentrations in the range of about 10⁶ to 10¹⁰ viruses per mL blood have been reported. In this case, the virus itself (constitutive protein(s)) of the virus is the exotoxin, and this explains the association between massive amounts of virus poured onto the blood and viral sepsis. Studies have found that many viral proteins have biological activities identical, or very similar, to those of bacterial exotoxins. Without being bound by any theory, it is believed that the evolutionary goal of the large (indeed, massive) virus concentrations is to suppress (or evade) host immune (inflammation) response and facilitate ongoing virus replication. Examination of proteins in viruses that cause sepsis shows that these viral proteins cause immunosuppression as well as epithelial barrier dysfunction and host cell apoptosis. The analogy with bacterial exotoxins is striking and almost certainly represents an example of parallel evolution (bacterial exotoxins and viral proteins with exotoxin function that evolved to produce similar effects). It is also proposed that free viral proteins in the circulation can increase exotoxin bioactivity that may be unexpected by measurement of viral genome alone.

In this disclosure, “epithelial barrier dysfunction” includes endothelial barrier dysfunction.

Unless defined otherwise, all technical and scientific terms used herein have the same meaning as commonly understood by one of ordinary skill in the art to which this disclosure belongs. Any methods and materials similar or equivalent to those described herein can be used in the practice or testing of the disclosure, as it will be understood that modifications and variations are encompassed within the spirit and scope of the instant disclosure. All publications mentioned herein are incorporated herein by reference in their entirety.

Specific embodiments disclosed herein may be further limited in the claims using “consisting of” or “consisting essentially of” language, rather than “comprising”. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

The terms “treat,” “treating,” “treatment,” and the like, including any grammatical variations thereof, refer to eliminating, reducing, relieving, reversing, and/or ameliorating a disease or condition and/or symptoms associated therewith, e.g., sepsis. Although not precluded, treating a disease or condition does not require that the disease, condition, or symptoms associated therewith be completely eliminated, including the treatment of acute or chronic signs, symptoms and/or malfunctions. Moreover, the term “treat” and synonyms contemplate administering a therapeutically effective amount of an active pharmaceutical ingredient (API) to an individual, e.g., a mammalian patient including, but not limited to, humans and/or veterinary animals, in need of such treatment. A treatment can be oriented symptomatically, for example, to suppress symptoms or severity of a disease or clinical condition associated with microorganism infection, e.g., sepsis. It can be affected over a short period, be oriented over a medium term, or can be a long-term treatment, for example within the context of a maintenance therapy.

As used herein, the terms “prophylaxis,” “prophylactic,” “prophylactically treating,” “prophylactic treatment,” “post-exposure prophylaxis” and the like, including any grammatical variations thereof, refers to reducing the probability of developing a disease or condition, e.g., sepsis, or of a recurrence of a previously-controlled disease or condition, in a subject who does not have, but is at risk of or is susceptible to, redeveloping a disease or condition or a recurrence of the disease or condition. Furthermore, the term “prophylaxis” and synonyms, including any grammatical variations thereof, contemplate administering a therapeutically effective amount of an active pharmaceutical ingredient (API) to an individual, e.g., a mammalian patient including, but not limited to, humans and/or veterinary animals, in need of preventing a disease or clinical condition, e.g., sepsis, or reducing the severity of any future development of a disease or clinical condition associated with microorganism infection, e.g., sepsis.

Unless explicitly stated or the context requires otherwise, the term “microorganism” means bacterium, mycobacterium, fungus, protozoan, virus, or a combination thereof. Exemplary microorganisms infections of which that can be treated using compositions and methods of the disclosure include, but are not limited to, any Gram-positive coccus (aerobic or anaerobic), including Pneumococcus (Streptococcus pneumoniae), Streptococcus groups A, B, C, or G, Streptococcus anginosus group (formerly Streptococcus milleri group=Streptococcus constellatus, Streptococcus intermedius, Streptococcus anginosus), Enterococcus faecalis and Enterococcus faecium, Staphylococci (coagulase negative and coagulase positive bacteria) including methicillin-sensitive Staphylococcus aureus (MSSA), methicillin-resistant Staphylococcus aureus (MRSA), vancomycin-resistant Staphylococcus aureus (VRSA), Staphylococcus epidermidis, Staphylococcus hominis, Leuconostoc; any Gram-positive rod or bacillus (aerobic or anaerobic), including Clostridia species (C. tetani or C. botulinum, C sordellii, or C. difficile), Listeria monocytogenes, Erysepelothrix, Bacillus species (Bacillus anthracis, Bacillus cereus); any Gram-negative coccus (aerobic or anaerobic), including Neisseriae, including Neisseria gonorrhea and Neisseria meningitis, Veillonella, Bartonella species including Bartonella henselae and Bartonella quintana, Moraxella catarrhalis; any Gram-negative rod or bacillus (aerobic or anaerobic) enteric or non-enteric pathogens including Pseudomonas species, bacteria like E. coli, Salmonella, Shigella, Campylobacter, Helicobacter pylori and Helicobacter cneidi, Proteus species including Proteus vulgaris and P. rettgeri, Eikenella corrodens, Aggegatibacter actinomycetemcomitans, Coxiella burnetti, Cardiobacterium hominis, Kingella kingae, Bacteroides fragilis, Serratia marcessans, Enterobacter cloacae, Citrobacter freundii, Morganella morganii, Providentia species, Haemophilus influenzae, Capnocytophaga canimorsus, Fusobacterium necrophorum and Fusobacterium nucleatum, Stenotrophomonas maltophilia, Streptobacillus moniliformis; any mycobacterium including Mycobacterium tuberculosis (TB) and non-tuberculous mycobacteria like Mycobacteria abscessus/chelonae/fortuitum/chimerae/bovis, Mycobacterium ulcerans, Mycobacterium marinum, Mycobacterium gordonae, Mycobacterium leprae; mycoplasma including Mycoplasma pneumoniae, Mycoplasma hominis; Actinomyces species including A. israelii/constillatus/naeslundii and Nocardia species; any protozoan pathogen including, Giardia lamblia, Entamoeba histolytica, Leishmania species (including L. donovani) Malaria (including species Malariae/Ovale/Falciparum/Vivax); any fungus including Candida (several species including albicans/tropicalis/glabrata/auris); any mold including Aspergillus (several species like fumigatus, flavus, niger, terreus), Mucorales, Rhizupus; dimorphic yeasts/molds including Histoplasma capsulatum, Coccidioides species (C. immitis or C. posadasii), Blastomyces dermatitidis, Paracoccidioides species; and any pathogenic virus including: Influenza viruses A, B, C, or D, Cytomegalovirus, Epstein-Bar Virus, Herpes viruses including Herpes simplex 1 and 2, Varicella-Zoster virus, Human Herpes virus 6, Severe Acute Respiratory Syndrome Coronavirus 2 (SARS-COV-2, agent that causes COVID-19), Ebola, Dengue, West Nile Virus, Respiratory syncytial virus, Parvovirus, Measles virus, BK virus, Crimean-Congo virus, Hantaviruses (including sin nombre), Human adenoviruses, JC virus.

Some aspects of the disclosure provide a method for treating or preventing (i.e., prophylaxis) of a disease or clinical condition associated with a microorganism infection. In one particular embodiment, the disease or clinical condition associated with microorganism infection is sepsis. The present inventor has discovered that such a treatment or prophylaxis can be achieved by heretofore unprecedented methods. In particular, some aspects of the disclosure provide a method for treating or preventing a clinical condition associated with microorganism infection by, for example, preventing development of or reducing production or activity of infecting microorganism's exotoxin(s).

One particular aspect of the disclosure provides a method for treating sepsis or other clinical conditions associated with a microorganism infection by using a therapeutic agent that (i) blocks ability of a microorganism to produce or release exotoxins; (ii) reduces, modulates, or prevents biological activity of exotoxin(s); (iii) preventing propagation of microorganism; or (iv) combination thereof. This includes expanded use of bacteriostatic (“static”) antibiotics that inhibit bacterial protein synthesis. Example antibiotics that can be used in methods of disclosure include, but are not limited to, oxazolidinones (linezolid), macrolides, and lincosamides. These kinds of drugs/antibiotics can be used synergistically with bactericidal “cidal” drugs/antibiotics that kill bacteria without any significant direct effect on reducing bacteria's exotoxin production. Still in another embodiment, methods of the disclosure provide treating sepsis or a clinical condition associated with microorganism infection by suppressing, preventing, or reducing the synthesis or export, delivery from the microorganism, or host cell uptake of exotoxins.

Another aspect of the disclosure provides a method for treating sepsis or a clinical condition associated with microorganism infection by blocking the adverse downstream physiological effects of exotoxins that produce sepsis. Such methods include administering a therapeutic agent that blocks biological activity of exotoxin. In another embodiment, methods of the disclosure block the adverse effects of exotoxin include using an immunoenhancing agent that augment immune function, e.g., exogenous pro-inflammatory cytokines, use of inhibitors of endogenous immune suppressor molecules such as inhibitors of IL-1 receptor antagonist, inhibitors of TNF soluble receptors, inhibitors of IL-10, or endogenous corticosteroid suppression. Enhancing innate immune function may kill or disable bacteria and therefore lower total exotoxin synthesis. It should be noted that this method is the opposite approach of conventional methods for treating sepsis. In another embodiment, the method includes using an agent, drug, or a compound that reverses or blocks exotoxin effects that are detrimental to the subject during sepsis or a clinical condition associated with microorganism infection. These include therapeutic agents that reverse or block epithelial barrier disruption or apoptosis.

Another aspect of the disclosure is suppression of the adverse effects of diffuse uncontrolled activation of the host-evolved dead man's switch device that normally functions to eliminate pathogen microorganisms. These measures include ameliorating production of long vWF strands bound to the surface of endothelial cells, or block attachment of platelets to the vWF strands of any combination thereof. Therapeutic agents that restore epithelial barrier function or that block apoptosis or that disrupt attachment of vWF strands to endothelial cells or inhibit platelet binding to these strands can also be used separately or in combination to treat sepsis or a clinical condition associated with microorganism infection.

Still in another embodiment, methods for treating sepsis or a clinical condition associated with microorganism infection include blocking the adverse physiological effects of exotoxins that produce sepsis or a clinical condition associated with microorganism infection. Such methods can include using an antibody, aptamer, resin, soluble receptor, proteolytic agent, enzyme inhibitor or other binding agent (either alone or in combination) that combine with and neutralize exotoxin biological effects. These drugs/agents can be administered intravenously, into the gut (e.g., oral consumption, use of a nasogastric tube, by rectal infusion or by tube insertion onto the gut percutaneously). Topical use in the upper airways is also a suitable application for subjects at risk for pneumonias or who are intubated. Topical, subcutaneous or transdermal methods of therapeutic agent delivery can also be used.

In another embodiment, methods for treating sepsis or a clinical condition associated with microorganism infection include blocking the adverse physiological effects of exotoxins that produce or cause sepsis or a clinical condition associated with microorganism infection. Such methods include using hemofiltration or hemodialysis-related measures that include filters or binding substances or other apparatus designed to remove exotoxins or viral proteins that function as exotoxins from blood.

Without being bound by any theory, it is believed that in certain cases, microorganisms release, produce or contain exotoxin precursors that require activity of host-derived enzymes to activate exotoxins. One particular example of such exotoxin includes anthrax lethal toxin and anthrax edema toxin. For these exotoxins to produce biological activity, there is a requirement for activity of a host cell-surface serine protease to activate the toxin. For these exotoxins, methods of the disclosure can include using an inhibitor of these host enzymes to block exotoxin activation. For example, use of the serine protease inhibitor alpha-1-antitrypsin to block anthrax exotoxin effect. Accordingly, in some embodiments, the therapeutic agent is an inhibitor of an enzyme that is required to activate exotoxin biological activity. A natural extension of this concept is blockade of exotoxin interaction with cell-surface receptor molecules that enable exotoxins to affect cells or to enter cells. Also, interruption of intracellular biological (signaling) activities that transmit signals that allow exotoxins to alter cell function is a further method.

Again without being bound by any theory, the present inventor has discovered that several exotoxins or molecules with similar function appear in the circulation simultaneously to cause sepsis or a clinical condition associated with microorganism infection during a single episode of sepsis or a clinical condition associated with microorganism infection in a patient. This is an expected consequence of the fact that single bacterial species can produce several exotoxins, and the observation that the initiating cause of sepsis or a clinical condition associated with microorganism infection can be polymicrobial infection. Viruses can contain more than one constitutive molecule(s) that possess(es) exotoxin function (FIG. 2). Also, exotoxin-induced gut hyperpermeability caused by the initial (inciting) infection or insult can result in several secondary or enhancing exotoxins to enter the circulation from within the gut. Exotoxins have been shown to be present in the gut during health and also during illness and these exotoxins can transmigrate into blood. The initial infection that secretes exotoxins into blood can increase epithelial barrier permeability of the gut and this can result in transmigration of gut-sequestered exotoxins into the circulation. This can establish a forward-feeding vicious cycle that incrementally increases transmigration of exotoxins in to the circulation and tissues. The human colon contains approximately 40 trillion bacteria. In some embodiments, the presence of multiple exotoxins in the infected patient will contribute to causing immunosuppression, and epithelial barrier dysfunction, and apoptosis. In such cases it would be beneficial to the patient to use two or more exotoxin inhibitors as combination therapy to treat sepsis or a clinical condition associated with microorganism infection. For example, one can use inhibitors of production of several exotoxins in the same patient to neutralize the effects of multiple exotoxins. Alternatively, or in addition, one can use a compound to block several independent steps that are required to generate exotoxin bioactivity. For example, use of bacteriostatic (protein synthesis inhibitor) antibiotics in combination with exotoxin binding resins or exotoxin-directed antibodies/soluble receptors and/or in combination with inhibitors of epithelial barrier disruption and/or in combination with apoptosis inhibitors and/or in combination with inhibitors of host-derived proteases that activate exotoxin activity.

Other aspects of the disclosure provide methods for preventing or immunizing (i.e., prophylaxis) against relevant exotoxins. Such methods will be beneficial and may have large societal impact. This method is particularly desired for reducing or preventing a post-operative microorganism infection in a subject. For example, by identifying the microorganism(s) or their exotoxins that are prevalent within a facility of scheduled operation or recovery, one can administer a vaccine against such microorganism(s) and/or a therapeutic agent that reduces, inhibits, or ameliorates the activity of exotoxins produced by such microorganism in order to reduce, prevent, or ameliorate incidences of sepsis or a clinical condition associated with microorganism infection. The therapeutic agent can be administered, for example, enterally, parenterally, or topically, prior to the operation.

Still another aspect of the disclosure provides a method for designing an immunization, vaccine, or a therapeutically active agent to treat or prevent a clinical condition associated with microorganism infection, e.g., relevant/important bacterial exotoxin(s) such as toxic-shock syndrome exotoxin, Shiga-like toxin from E. coli, Pseudomonas exotoxin A, Pneumococccal pneumolysin, phenol soluble modulin-like proteins, etc. These can be administered as an immunization directed against a single toxin or a combination (“cocktail”) vaccination against several exotoxins (like the Tdap, which contains toxoid derived from tetanus toxin, diphtheria toxin, and pertussis toxin). Vaccines against bacterial or viral exotoxins can be constructed by those skilled in the art with specific immune responses produced to protect specific populations at risk for certain infections. This includes immunocompromised patients or those of advanced age or with medical co-morbidities.

Yet another aspect of the disclosure provides a method for designing an immunization that protect against sepsis or a clinical condition associated with microorganism infection caused by pathogen(s) separate from the pathogens whose exotoxins are used for immunization. It has been observed that some vaccinations against one pathogen can cross-protect against one or more separate pathogens. For instance, the BCG (Bacille Calmette Guerin) vaccination used in Europe (a Tuberculosis vaccine) also protects persons who are vaccinated against infections caused by different microbes. The mechanism of cross-protection is thought to originate from “trained innate immunity” or enhanced immune response to several types of infecting microbes. Similarly, some aspects of the disclosure use exotoxin directed vaccinations to provide cross-protection against several separate exotoxins. This aspect of the disclosure possesses additional rationale since exotoxins naturally separate into families of structurally similar toxins (for example, superantigens, pore-forming toxins, cholesterol-dependent cytolysins, metalloproteases, repeat-in-toxin exotoxins, etc.). Therefore, immunization directed against one toxin can be used to provide protection against several additional exotoxins (especially exotoxins from the same family of toxins). Therefore, immunization against one exotoxin will likely afford broad protection against other exotoxins in the same family as the original toxin used to generate immunity.

Another aspect of the disclosure provides a method for treating heatstroke. Heatstroke is a clinical syndrome caused by exposure to excessive heat that overwhelms the body's thermoregulatory system. In this case, heatstroke presents clinically (at the bedside) very similarly to sepsis. The parallels were considered so similar (heatstroke and sepsis), that excessive inflammation was thought to be a cause of heatstroke. Following this pathway of belief, steroids were tried in heatstroke patients; these studies failed to show benefit (similar to the failure of steroids to treat sepsis; see TABLE 1). As in the case of sepsis, the present inventor has discovered that heatstroke is caused at least in part by exotoxin storm, i.e., presence of an excess amount of exotoxins that disseminate widely though the body via access to the circulation. Studies have shown that heatstroke causes excessive gut permeability and that enormous amounts of endotoxin (e.g., surface components of Gram-negative bacteria) pour into the blood. While the endotoxins do not cause the heatstroke, it is believed that endotoxin translocation from gut into blood is accompanied by substantial transfer of bacterial exotoxins into the blood (in this case the translocation of endotoxin is a marker for concomitant transfer of exotoxins into the blood; exotoxin molecules are in general smaller than endotoxin molecules). This series of events reflects similar pathogenesis mechanisms for heatstroke as for sepsis and accounts for the similar clinical presentations (heatstroke and sepsis) and the absence of beneficial effects of steroids. Therefore, therapeutic measures described above to treat sepsis can also be used in treating heatstroke.

There are special circumstances where use of anti-inflammatory agents/drugs (that have all failed during natural sepsis) should benefit a patient with severe infection. One such circumstance is Jarisch-Herxheimer reaction (JHR). This represents a condition where anti-inflammatory measures are believed to be beneficial. JHR occurs when infections with spirochete bacteria are treated with antibiotics. The two prominent examples include infections with the spirochete Treponema pallidum (Syphilis) and spirochetes that cause Relapsing Fever infections. Additional examples include spirochetes that cause Leptospirosis and Lyme disease. In these cases, there is first and foremost no exotoxin-induced immunosuppression, since these bacteria do not have exotoxins, as established by prior art sequencing of the genomes of these bacteria. Exotoxin-related genetic code was absent. To survive, spirochete bacteria have evolved mechanisms to evade (not suppress) inflammation by cloaking themselves with outer surface molecules that are poorly recognized by the immune system, or by rapidly altering the surface bacterial components (a “moving target” that is difficult for the immune system to keep targeting). However, antibiotic-induced destruction of bacteria exposes many internal bacterial parts/components to host immune cells that then generate an exuberant immune response, i.e., an actual cytokine storm. This is a somewhat artificial circumstance brought about by use of antibiotics and is thus not a part of the evolutionary history of natural sepsis. These spirochete bacteria do not produce exotoxins. Therefore, there is no immune suppression that the bacterium generates during infection. Unlike exotoxin-caused sepsis, these spirochete bacteria do not cause true sepsis (there is no immunosuppression, and immune suppression occurs during natural sepsis). The JHR caused by antibiotic treatment is a rare example of real cytokine storm or hyper-immunity in the context of an infection. However, this is not a natural phenomenon, since intervention (antibiotic destruction of many bacteria) is required to produce the host excessive inflammatory response.

Another example where an infectious complication can benefit from anti-inflammatory drugs/agents is inadvertent infusion of an intravenous solution that is contaminated with bacteria or virus particles. In this case a large load of bacterial or viral components (e.g., endotoxins in the case of Gram-negative bacteria or lipoteichoic acid (LTA) in the case of Gram-positive bacteria) is infused as a bolus infusion directly into the bloodstream rapidly and in the absence of a large load of exotoxins. In this circumstance, a hyper-inflammatory (cytokine storm) state can rapidly arise and be treated with anti-inflammatory/anti-cytokine therapies. The rationale is rapid infusion of inflammation-inducing endotoxin and/or lipoteichoic acid (as well as other pathogen components) that are recognized by the innate immune system (sensed by host immune cells) in the absence of counteracting immune-suppressing exotoxins. This recognition generates inflammation before the pathogen has an opportunity to synthesize sufficient exotoxin(s) to suppress the innate immune (inflammation) response. This scenario presupposes absent or low synthesis of exotoxins in bacteria that contaminate the infusion or blood product. There are numerous examples that show that bacteria-synthesized exotoxins are inducible. Importantly, bacterial exotoxin production is upregulated in the presence of stimuli that are encountered after invasion of the host. Examples of such stimuli include host-derived cytokines themselves (in particular TNF), presence of immune cells, antibiotics, epinephrine, bacterial crowding (exotoxins are induced by bacterial quorum sensing) and others. Therefore, exotoxin levels will be absent or small at the time of initial infusion of bacteria into the bloodstream. Another reason for low exotoxin levels in the infusion liquid is the low temperatures that blood products are stored at. This is likely due to cold-induced low metabolic activity of the bacteria that will retard microbial synthesis of exotoxins. After bacteria are infused into the host, the bacteria will replicate and sense the presence of the host (they are then inside the host body) and begin to synthesize exotoxins. But this event will likely be delayed until after the host immune cells recognize the bacterial invasion and generate an immune (inflammation) response. Bacterial exotoxin synthesis will also be aborted after infusion of antibiotics, and antibiotic therapy would be administered immediately after infusion of a contaminated blood product is recognized.

Other aspects of the disclosure provide methods for producing antibody to the exotoxin. Useful antibodies of the disclosure can include serum containing such antibodies, or antibodies that have been purified to varying degrees. Whole antibodies of the disclosure can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), can also be employed in the disclosure.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies can be produced according to the methodology of Kohler and Milstein (Nature, 1975, 256, 495-497). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

Therapeutic agents and methods of the present disclosure are also applicable for treating any clinical conditions that is caused by epithelial or endothelial barrier disruption where exotoxin or constitutive exotoxin-functioning viral proteins are culprits. Exemplary clinical conditions that are associated with epithelial or endothelial barrier disruption include, but are not limited to, sepsis, heatstroke, hypotension/hypoperfusion that results in epithelial or endothelial barrier disruption, blood loss that results in epithelial or endothelial barrier disruption, trauma that leads to epithelial or endothelial barrier disruption, typhlitis (e.g., as a result of chemotherapy), JHR, substantial infusion of bacteria or endotoxin or lipoteichoic acid. In one particular embodiment of the disclosure provides use of a therapeutic agent, such as, but not limited to, antibody, aptamer, recombinant ADAMTS-13, a plasma composition comprising ADAMTS-13 or natural or synthetic substance with ADAMTS-13 protease activity, an inhibitor that reduces production of or inhibits binding of von Willebrand Factor (vWF) strands to the surface of endothelial cell P-selectin, an inhibitor that reduces or inhibits binding of platelets to vWF strands (e.g., CAPLACIZUMAB, trade name Cablivi®), an aptamer that selectively binds to and inactivates or clears said exotoxin, or a combination thereof. Such a therapeutic agent can be used to treat heatshock, sepsis, septic shock, trauma, blood loss, typhlitis, Clostridioides difficile disease, shiga-like toxin mediated diarrhea, hemolytic-uremic syndrome (HUS), immune reconstitution inflammatory syndrome, disseminated intravascular coagulopathy (DIC), purpura fulminans, necrotizing fasciitis, hemorrhagic fever, or a combination thereof. One particular embodiment of the disclosure provides use of a therapeutic agent for treating sepsis.

Another aspect of the disclosure provides use of an antibody to the exotoxin as a therapeutic agent. As used herein, the term “antibody molecule” refers to a protein comprising at least one immunoglobulin variable domain sequence. The term antibody molecule includes, for example, full-length, mature antibodies and antigen-binding fragments of an antibody. For example, an antibody molecule can include a heavy (H) chain variable domain sequence (abbreviated herein as VH), and a light (L) chain variable domain sequence (abbreviated herein as VL). In another example, an antibody molecule includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. In one aspect, functional antibody fragments retain the ability to selectively bind with their respective antigen or receptor. In various embodiments, antibodies and antibody fragments can be from any class of antibodies including, but not limited to, IgG, IgA, IgM, IgD, and IgE, and from any subclass (e.g., IgG1, IgG2, IgG3, and IgG4) of antibodies, and can be monoclonal or polyclonal, human, humanized, CDR-grafted, or an in vitro generated antibody. In other aspects, the antibody can have a heavy chain constant region chosen from, e.g., IgG1, IgG2, IgG3, or IgG4. In another embodiment, the antibody can have a light chain chosen from, e.g., kappa or lambda.

Examples of antigen-binding fragments include: (i) a Fab fragment, a monovalent fragment consisting of the VL, VH, CL and CH1 domains; (ii) a F(ab′) 2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); (viii) a single domain antibody. In one aspect, these antibody fragments can be obtained using conventional techniques known to those with skill in the art, and the fragments can be screened for utility in the same manner as are intact antibodies.

The term “antibody” includes intact molecules as well as functional fragments thereof. In various embodiments, constant regions of the antibodies can be altered, e.g., mutated, to modify the properties of the antibody (e.g., to increase or decrease one or more of: Fc receptor binding, antibody glycosylation, the number of cysteine residues, effector cell function, or complement function).

In one aspect, antibody molecules can also be single domain antibodies. Single domain antibodies can include antibodies whose complementary determining regions are part of a single domain polypeptide. Examples include, but are not limited to, heavy chain antibodies, antibodies naturally devoid of light chains, single domain antibodies derived from conventional 4-chain antibodies, engineered antibodies and single domain scaffolds other than those derived from antibodies. Single domain antibodies may be any known in the art, or any future single domain antibodies. In various embodiments, single domain antibodies may be derived from any species including, but not limited to mouse, human, camel, llama, fish, shark, goat, rabbit, and bovine. According to another aspect of the invention, a single domain antibody is a naturally occurring single domain antibody known as heavy chain antibody devoid of light chains. Such single domain antibodies are disclosed in WO 9404678, for example. For clarity, this variable domain derived from a heavy chain antibody naturally devoid of light chain is described herein as a VHH or nanobody to distinguish it from the conventional VH of four chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

The VH and VL regions can be subdivided into regions of hypervariability, termed “complementarity determining regions” (CDR), interspersed with regions that are more conserved, termed “framework regions” (FR or FW).

The extent of the framework region and CDRs has been precisely defined by a number of methods (see, Kabat, E. A., et al. (1991) Sequences of Proteins of Immunological Interest, Fifth Edition, U.S. Department of Health and Human Services, NIH Publication No. 91-3242; Chothia, C. et al. (1987) J. Mol. Biol. 196:901-917; and the AbM definition used by Oxford Molecular's AbM antibody modeling software. See, generally, e.g., Protein Sequence and Structure Analysis of Antibody Variable Domains. In: Antibody Engineering Lab Manual (Ed.: Duebel, S. and Kontermann, R., Springer-Verlag, Heidelberg).

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme). As used herein, the CDRs defined according the “Chothia” number scheme are also sometimes referred to as “hypervariable loops.”

For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may or may not include one, two, or more N- or C-terminal amino acids, or may include other alterations that are compatible with formation of the protein structure.

The term “antigen-binding site” refers to the part of an antibody molecule that comprises determinants that form an interface that binds to the antigen, or an epitope thereof. With respect to proteins (or protein mimetics), the antigen-binding site typically includes one or more loops (of at least four amino acids or amino acid mimics) that form an interface that binds to the antigen. Typically, the antigen-binding site of an antibody molecule includes at least one or two CDRs and/or hypervariable loops, or more typically at least three, four, five or six CDRs and/or hypervariable loops.

The terms “monoclonal antibody” or “monoclonal antibody composition” as used herein refer to a preparation of antibody molecules of single molecular composition. A monoclonal antibody composition displays a single binding specificity and affinity for a particular epitope. A monoclonal antibody can be made by hybridoma technology or by methods that do not use hybridoma technology (e.g., recombinant methods).

In various embodiments, the antibody molecule can be a polyclonal or a monoclonal antibody. In other embodiments, the antibody can be recombinantly produced, e.g., produced by phage display or by combinatorial methods.

Phage display and combinatorial methods for generating antibodies are known in the art (as described in, e.g., Ladner et al. U.S. Pat. No. 5,223,409; Kang et al. International Publication No. WO 92/18619; Dower et al. International Publication No. WO 91/17271; Winter et al. International Publication WO 92/20791; Markland et al. International Publication No. WO 92/15679; Breitling et al. International Publication WO 93/01288; McCafferty et al. International Publication No. WO 92/01047; Garrard et al. International Publication No. WO 92/09690; Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982, the contents of all of which are incorporated by reference herein).

In one embodiment, the antibody is a fully human antibody (e.g., an antibody made in a mouse, rabbit, porcine, primate, chicken, or other animals which has been genetically engineered to produce an antibody from a human immunoglobulin sequence), or a non-human antibody, e.g., a rodent (mouse or rat), goat, primate (e.g., monkey), camel antibody. In one aspect, the non-human antibody is a rodent (mouse or rat antibody). Methods of producing rodent antibodies are known in the art.

Human monoclonal antibodies can be generated using transgenic mice or other suitable animals carrying the human immunoglobulin genes rather than the animal's own system. For example, splenocytes from these transgenic mice immunized with the antigen of interest can be used to produce hybridomas that secrete human mAbs with specific affinities for exotoxins.

In one aspect, an antibody can be one in which the variable region, or a portion thereof, e.g., the CDRs, are generated in a non-human organism, e.g., a rat or mouse. In another aspect, chimeric, CDR-grafted, and humanized antibodies can be used. In another embodiment, antibodies generated in a non-human organism, e.g., a rat or mouse, and then modified, e.g., in the variable framework or constant region, to decrease antigenicity in a human can be used.

Chimeric antibodies can be produced by recombinant DNA techniques known in the art (see Robinson et al., International Patent Publication PCT/US86/02269; Akira, et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al. (1988 Science 240:1041-1043); Liu et al. (1987) PNAS 84:3439-3443; Liu et al., 1987, J. Immunol. 139:3521-3526; Sun et al. (1987) PNAS 84:214-218; Nishimura et al., 1987, Canc. Res. 47:999-1005; Wood et al. (1985) Nature 314:446-449; and Shaw et al., 1988, J. Natl Cancer Inst. 80:1553-1559).

In one embodiment, a humanized or CDR-grafted antibody can have at least one or two but generally all three recipient CDRs (of heavy and or light immuoglobulin chains) replaced with a donor CDR. In various aspects, the antibody may be replaced with at least a portion of a non-human CDR or only some of the CDRs may be replaced with non-human CDRs. In one illustrative embodiment, the number of CDRs required for binding of the humanized antibody to the antigen can be replaced. In one aspect, the donor can be a rodent antibody, e.g., a rat or mouse antibody, and the recipient can be a human framework or a human consensus framework. Typically, the immunoglobulin providing the CDRs is called the “donor” and the immunoglobulin providing the framework is called the “acceptor.” In one embodiment, the donor immunoglobulin is a non-human (e.g., rodent). In one exemplary embodiment, the acceptor framework is a naturally-occurring (e.g., a human) framework or a consensus framework, or a sequence about 85% or higher, or with about 90%, 95%, 96%, 97%, 98%, 99% or higher identity thereto.

As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus framework” refers to the framework region in the consensus immunoglobulin sequence.

An antibody can be humanized by methods known in the art (see, for example, Morrison, S. L., 1985, Science 229:1202-1207, by Oi et al., 1986, BioTechniques 4:214, and by Queen et al. U.S. Pat. Nos. 5,585,089, 5,693,761 and 5,693,762, the contents of all of which are hereby incorporated herein by reference).

Humanized or CDR-grafted antibodies can be produced by CDR-grafting or CDR substitution, wherein one, two, or all CDRs of an immunoglobulin chain can be replaced. See e.g., U.S. Pat. No. 5,225,539; Jones et al. 1986 Nature 321:552-525; Verhoeyan et al. 1988 Science 239:1534; Beidler et al. 1988 J. Immunol. 141:4053-4060; Winter U.S. Pat. No. 5,225,539, the contents of all of which are hereby expressly incorporated by reference. Winter describes a CDR-grafting method which may be used to prepare humanized antibodies used in the combinations and methods described herein (UK Patent Application GB 2188638A, filed on Mar. 26, 1987; Winter U.S. Pat. No. 5,225,539), the contents of which is expressly incorporated by reference herein.

In additional aspects, humanized antibodies in which specific amino acids have been substituted, deleted or added can be used. Criteria for selecting amino acids from the donor are described in U.S. Pat. No. 5,585,089, e.g., columns 12-16 of U.S. Pat. No. 5,585,089, e.g., columns 12-16 of U.S. Pat. No. 5,585,089, the contents of which are hereby incorporated by reference herein. Other techniques for humanizing antibodies are described in Padlan et al. EP 519596 A1, published on Dec. 23, 1992.

In one embodiment, the antibody molecule can be a single chain antibody. In one aspect, a single-chain antibody (scFV) may be engineered (see, for example, Colcher, D. et al. (1999) Ann N Y Acad Sci 880:263-80; and Reiter, Y. (1996) Clin Cancer Res 2:245-52). In another aspect, the single chain antibody can be dimerized or multimerized to generate multivalent antibodies having specificities for different epitopes of the same target protein.

In yet other embodiments, the antibody molecule has a heavy chain constant region chosen from, e.g., the heavy chain constant regions of IgG1, IgG2, IgG3, IgG4, IgM, IgA1, IgA2, IgD, and IgE; or chosen from, e.g., the (e.g., human) heavy chain constant regions of IgG1, IgG2, IgG3, and IgG4. In another embodiment, the antibody molecule has a light chain constant region chosen from, e.g., the (e.g., human) light chain constant regions of kappa or lambda. In another aspect, the constant region can be altered, e.g., mutated, to modify the properties of the antibody (e.g., to increase or decrease one or more of: Fc receptor binding, antibody glycosylation, the number of cysteine residues, effector cell function, and/or complement function). In one embodiment, the antibody has effector function and can fix complement. In other embodiments, the antibody does not recruit effector cells or fix complement. In another embodiment, the antibody has reduced or no ability to bind an Fc receptor. For example, it is an isotype or subtype, fragment or other mutant, which does not support binding to an Fc receptor, e.g., it has a mutagenized or deleted Fc receptor binding region.

Methods for altering an antibody constant region are known in the art. In one embodiment, antibodies with altered function, e.g., altered affinity for an effector ligand, such as FcR on a cell, or the Cl component of complement can be produced by replacing at least one amino acid residue in the constant portion of the antibody with a different residue (see e.g., EP 388,151 A1, U.S. Pat. Nos. 5,624,821 and 5,648,260, the contents of all of which are hereby incorporated by reference). Similar types of alterations could be used which if applied to the murine, or other species immunoglobulin would reduce or eliminate these functions.

The foregoing discussion of the disclosure has been presented for purposes of illustration and description. The foregoing is not intended to limit the disclosure to the form or forms disclosed herein. Although the description of the disclosure has included description of one or more embodiments and certain variations and modifications, other variations and modifications are within the scope of the disclosure, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A method for treating a subject for a clinical condition associated with a microorganism infection, said method comprising administering to a subject in need of such a treatment a therapeutically effective amount of a therapeutic agent capable of reducing the effect of an exotoxin or exotoxin-functioning constitutive molecule associated with said microorganism, thereby treating said subject.

2. The method of claim 1, wherein said microorganism comprises bacterium, mycobacterium, fungus, protozoan, virus, or a combination thereof.

3. The method of claim 1, wherein said clinical condition comprises heatstroke or heatshock, sepsis, septic shock, trauma, blood loss, typhlitis, Clostridioides difficile disease, shiga-like toxin mediated diarrhea, hemolytic-uremic syndrome (HUS), immune reconstitution inflammatory syndrome, disseminated intravascular coagulopathy (DIC), purpura fulminans, necrotizing fasciitis, hemorrhagic fever, or a combination thereof.

4. The method of claim 1, wherein said clinical condition comprises sepsis.

5. The method of claim 1, wherein said therapeutic agent comprises a plurality of agents each of which is capable of reducing the effect of a different exotoxin or constitutive molecule or molecules with exotoxin function of said microorganism.

6. The method of claim 1, wherein said therapeutic agent comprises a neutralizing antibody directed against said exotoxin, antitoxin neutralizing resin, anti-serum comprising an antibody directed against said exotoxin, an aptamer that is capable of neutralizing exotoxins or exotoxin-functioning constitutive molecules.

7. The method of claim 1, wherein said therapeutic agent comprises an antagonist or an inhibitor of a cell-surface receptor or intracellular receptor that binds with said exotoxin.

8. The method of claim 1, wherein said therapeutic agent comprises an antibody directed against said exotoxin.

9. The method of claim 1, wherein said therapeutic agent increases the level of or the activity of a Toll-Like Receptor(s) (TLR), inflammasome(s), retinoic-acid inducible gene 1 (RIG 1)-like receptor(s), C-type-lectin-like receptor(s), or a combination thereof.

10. The method of claim 1, wherein said therapeutic agent comprises a recombinant human ADAMTS-13, a purified ADAMTS-13, or a plasma or other blood-derived preparation that comprises an endogenous ADAMTS-13.

11. The method of claim 1, wherein said therapeutic agent comprises CAPLACIZUMAB® or CABLIV™ monoclonal antibody.

12. A method for treating a disease or a clinical condition caused by a microorganism infection in a patient, said method comprising:
identifying the microorganism causing the disease or the clinical condition in the patient; and
administering to the patient a therapeutically effective amount of a therapeutic agent capable of reducing the effect of one or more exotoxins produced by said microorganism, thereby treating the disease or the clinical conditions caused by the microorganism infection in the patient.

13. The method of claim 12, wherein said therapeutic agent comprises an antibody directed against said exotoxin, antitoxin resin, an inhibitor of said exotoxin, anti-serum containing an antibody to said exotoxin, an aptamer that neutralizes biological activity of said exotoxin, or a combination thereof.

14. The method of claim 12, wherein said clinical condition comprises sepsis, septic shock, trauma, blood loss, typhlitis, Clostridioides difficile disease, shiga-like toxin mediated diarrhea, hemolytic-uremic syndrome, immune reconstitution inflammatory syndrome, disseminated intravascular coagulopathy (DIC), purpura fulminans, necrotizing fasciitis, hemorrhagic fever, or a combination thereof.

15. The method of claim 12, wherein said clinical condition comprises sepsis.

16. A method for producing a therapeutic agent for treating a microorganism infection in a subject, said method comprising:
identifying one or more exotoxin(s) associated with the microorganism; and
producing a therapeutic agent that inhibits activity of said exotoxin(s).

17. The method of claim 16, wherein said therapeutic agent comprises an aptamer, an antibody, or a combination thereof.

18. The method of claim 17, wherein said antibody is monoclonal antibody.

19. The method of claim 17, wherein said antibody is polyclonal antibody.

20. The method of claim 16, wherein said microorganism comprises a bacterium, a mycobacterium, a protozoan, a virus, a fungus, or some combination thereof.
